(19) United States
(12) Patent Application Publication
(10) Pub. No.: US 2022/0177525 A1
(43) Pub. Date: Jun. 9, 2022

(54) NOVEL LYSSA VIRUS PHOSPHOPROTEINS

(71) Applicant: The University of Melbourne, Victoria (AU)

(72) Inventors: Paul Raymond GOOLEY, Victoria (AU); Gregory William MOSELEY, Victoria (AU); Alamgir HOSSAIN, Victoria (AU)

(21) Appl. No.: 17/600,757

(22) PCT Filed: Aug. 27, 2019
(86) PCT No.: PCT/AU2019/050908

§ 371 Date: Oct. 1, 2021

(30) Foreign Application Priority Data
Apr. 3, 2019 (AU) 2019901137

(51) Int. Cl. C07K 14/005; C12N 7/00; A61K 39/205

(52) U.S. Cl. CPC C07K 14/005; C12N 7/00; A61K 39/205; A61K 2039/522; C12N 2760/20122; C12N 2760/20134; C12N 2760/20121

(57) ABSTRACT

The present invention provides an isolated lyssavirus phosphoprotein (P-protein) comprising one or more amino acid substitutions in a signal transducer and activator of transcription 1 (STAT1) interacting surface of the P-protein.

This application claims priority from Australian Provisional Patent Application No. 2019901137 filed on 3 Apr. 2019, the contents of which are to be taken as incorporated herein by this reference.

### FIELD OF THE INVENTION

The present invention is generally related to Lyssaviruses that have modified virulence and methods for making and using them, including Lyssavirus polypeptides, virions, immune stimulating compositions, and methods for the treatment and/or prevention of Lyssavirus infection, including rabies virus infection.

### BACKGROUND

Rabies is an untreatable disease of humans, which has a case-fatality rate of almost 100% in non-vaccinated individuals. The etiological agents of rabies are viruses of the almost globally distributed Lyssavirus genus, the best characterized of which is rabies virus (RABV) that infects diverse mammalian species with transmission to humans most commonly through bites from infected dogs.

The Rhabdoviridae family includes the Lyssavirus genus of viruses, which includes rabies virus, Lagos bat virus (LBV), Mokola virus (MOKV), Duvenhage virus (DUVV), European bat lyssavirus-1 (EBLV-1), European bat lyssavirus-2 (EBLV-2), Australian bat lyssavirus (ABLV), Aravan virus (ARAV), Khujand virus (KHUV), Irkut virus (IRKV), West Caucasian bat virus (WCBV) and Shimoni bat virus.

The development of new and improved vaccines is a priority for the control of rabies and other lyssaviruses, especially in animal populations, which is recognised as a highly effective approach to preventing human disease.

Although inactivated rabies vaccines prepared from cell culture are safe and well-tolerated, they have multiple disadvantages. They are difficult to manufacture, difficult to store, have low immunogenicity, and require multiple injections. Moreover, they are expensive and thus beyond the reach of most people who need to use the vaccines, such as in developing countries. In addition, these inactivated vaccines typically include adjuvants which may cause unwanted side effects.

Because human rabies is a zoonotic disease, there is a reliance on control of animals with the disease, such as catching stray dogs and vaccinating parenterally. While baits containing oral vaccines have been demonstrated to work for wild animals, there are safety and efficacy issues with attenuated oral vaccines for dogs which prevent effective bating campaigns. Thus, safer, cheaper, and more efficacious vaccines are needed.

The principal host-cell response to viral infection is activation of the innate immune response, and lyssaviruses such as rabies virus are able to counter these innate immune responses.

There remains a need to develop effective immune-stimulating lyssavirus vaccines.

### SUMMARY OF THE INVENTION

The present inventors have characterised a signal transducer and activator of transcription 1 (STAT1) interacting surface of the lyssavirus P-protein. Accordingly, in one aspect the present invention provides an isolated lyssavirus phosphoprotein (P-protein) comprising one or more amino acid substitutions in a STAT1 interacting surface of the P-protein.

The present inventors have characterised the STAT1 interacting surface, which is within the C-terminal domain (CTD) of the P-protein. Accordingly, in one embodiment the present invention provides an isolated lyssavirus P-protein as described herein, wherein the STAT1 interacting surface is within the C-terminal domain (CTD) of the P-protein. In another embodiment, the interacting surface is within the region corresponding to residues 186 to 297 of SEQ ID NO: 1.

In one aspect the present invention provides an isolated lyssavirus P-protein as described herein, wherein the one or more amino acid substitutions is within, or adjacent to, α helix 1, α helix 2 and/or α helix 5 of the of the C-terminal domain of the P-protein, for example, as shown in FIG. 1.

In one aspect the present invention provides an isolated lyssavirus P-protein as described herein, wherein the one or more amino acid substitutions interferes with the interaction of the P-protein with STAT1.

Importantly, the present inventors have demonstrated that one or more amino acid substitutions can modulate IFN antagonistic activity of the P-protein. Accordingly, in one aspect the present invention provides an isolated lyssavirus P-protein as described herein, wherein the one or more amino acid substitutions modulates IFN antagonistic activity of the P-protein.

In one aspect the present invention provides an isolated lyssavirus P-protein as described herein, wherein the P-protein does not comprise an amino acid substitution in the W-hole of the P-protein.

In one aspect, the present invention provides an isolated lyssavirus P-protein as described herein, wherein the one or more amino acid substitutions do not abolish polymerase cofactor function. In another preferred embodiment the one or more acid substitutions allow viral transcription, and/or allow viral replication, and/or allow binding to N protein.

In one aspect the present invention provides an isolated lyssavirus P-protein as described herein, wherein the one or more amino substitutions are not in the N-protein interacting surface.

In another aspect the present invention provides an isolated lyssavirus P-protein as described herein, wherein the one or more amino acid substitutions are in the region corresponding to amino acid residues 203 to 277 of SEQ ID NO: 1.

In another aspect the present invention provides an isolated lyssavirus P-protein as described herein, wherein the one or more amino acid substitutions are at an amino acid residue corresponding to amino acid residues 203, 204, 206, 207, 209, 234, 235, 236, 239 and/or 277 of SEQ ID NO: 1

In another aspect the present invention provides an isolated lyssavirus P-protein as described herein, wherein the one or more amino acid substitutions are at an amino acid residue corresponding to amino acid residues 203, 204, 206, 207, 209, 234, 235, 236, 239 and/or 277 of SEQ ID NO: 1, wherein the substitutions are 203A, 206G, 207A, 209A, 234A, 235A, 235K, 236A, 239A, and/or 277A.

In another aspect the present invention provides an isolated lyssavirus P-protein as described herein, wherein the P protein comprises at least two amino acid substitutions.

In another aspect the present invention provides an isolated lyssavirus P-protein as described herein, wherein the, wherein the at least two amino acid substitutions are at amino acid residues selected from the group consisting of amino acid residues corresponding to amino acid residues 206, 209 and 235 of SEQ ID NO: 1. In another embodiment, the present invention provides an isolated lyssavirus P-protein as described herein, wherein the two amino acid substitutions are selected from the group consisting of F209A, D235A, A206E, D235K and D236A, or an equivalent conserved position.

In another aspect the present invention provides an isolated lyssavirus P-protein as described herein, further comprising one or more amino acid substitutions in the W-hole of the P-protein.

In another aspect the present invention provides an isolated lyssavirus P-protein as described herein, wherein one or more amino acid substitutions are at an amino acid residue corresponding to amino acid residue 265 and/or or 287 of SEQ ID NO: 1.

In another aspect the present invention provides an isolated lyssavirus P-protein as described herein, wherein the amino acid substitutions are 265G or 287V, or an equivalent conserved position.

In another aspect the present invention provides an isolated lyssavirus P-protein as described herein, wherein the lyssavirus is selected from the group consisting of rabies virus, Lagos bat virus, Mokola virus, Duvenhage virus, European Bat lyssaviruses 1 and 2, Irkut virus, West Caucasian bat virus and Australian bat lyssavirus.

In one aspect, the present invention provides an isolated nucleic acid encoding a P-protein as described herein, or a complement thereof.

In another aspect, the present invention provides a cell or vector comprising a nucleic acid as described herein.

In another aspect, the present invention provides a lyssavirus genome, wherein the complement of the lyssavirus genome encodes a P-protein as described herein.

In another aspect, the present invention provides a lyssavirus virion comprising a lyssavirus genome as described herein. In a preferred embodiment, the present invention provides a lyssavirus virion comprising a lyssavirus genome as described herein, wherein the lyssavirus is attenuated. In another preferred embodiment, the present invention provides a lyssavirus virion comprising a lyssavirus genome as described herein, wherein the lyssavirus is able to replicate.

In another aspect, the present invention provides a pharmaceutical composition comprising a lyssavirus virion as described herein, and a pharmaceutically acceptable carrier.

In another aspect, the present invention provides a use of a lyssavirus virion as described herein in the manufacture of a medicament for treating and/or preventing lyssavirus infection in a subject.

In another aspect, the present invention provides a method of treating and/or preventing lyssavirus infection in a subject, said method comprising administering to the subject a therapeutically effective amount of a virion according as described herein or a pharmaceutical composition as described herein.

### BRIEF DESCRIPTION OF THE DRAWINGS

FIG. 1: Characterisation of a signal transducer and activator of transcription 1 (STAT1) interacting surface of the P-protein.

(a) The intensity ratios of the amide proton resonances observed from ¹⁵N, ¹H TROSY of NiP-CTD in the presence and absence of irradiation are plotted against residue numbers and secondary structure. The intensity ratio shown in black lines (circles) are for the apo-form (no STAT1) of NiP-CTD while light grey (squares) and dark grey (diamonds) lines are in the presence of GB1-STAT1-CCD-DBD and GB1-STAT1 respectively. In the presence of full-length GB1-STAT1 the intensity for H203, 1205, A206, E207 and D235 reduces to <0.5 (full line) while reduction of intensity for 1201, Q204, F209, D236, 1237, L276 and L277 are within the 0.6-0.5 range (dashed line). Residues contributing to the interactions lie within three regions: region A (Glu200 to Ser210), region B (Leu234 to Lys239) and region C (GIn275 to Val278), corresponding to helix 1, helix 2 and helix 5 of NiP-CTD. W265 and M287 of the W-hole are also indicated. (b) Surface representations of residues significantly attenuated (intensity ratio <0.6) in the transferred cross-saturation with full-length GB1-STAT1 are mapped onto the crystal structure of the CTD of P protein from the CVS strain (pdb: 1vyi). NH of residues that lose intensity <0.5 are shown in double circle, those within 0.6-0.5 range are in dashed circle. All significantly affected residues appear to lie on the round face of the protein (left panel). In contrast W265 and M287, shown in full circle, lie on the flat face of NiP-CTD (right panel).

FIG. 2: Multiple sequence alignment of Lyssavirus P proteins.

Shown is a CLUSTAL W 2.1 sequence alignment of RABV (P: Q9IPJ8), ABLV (P: Q8JTH2), EBLV1 (P: A4UHP9), EBLV2 (P: A4UHQ4), DUVV (P: 056774), IRKV (P: Q5VKP5), ARAV (P: Q6X1D7), KHUV (P: Q6X1D3), MOKV (P: P0C569), LBV (P: O56773), and WBCV (P: Q5VKP1). “*” denotes positions that have a single and fully conserved residue, “:” denotes conservation between residues of strongly similar properties with a score greater than 0.5 on the PAM 250 matrix, and “.” Denotes conservation between residues of weakly similar properties with a score less than or equal to 0.5 on the PAM 250 matrix.

FIG. 3: Amino acid substitutions in the STAT1 interacting surface of the P-protein do not abolish binding to N protein.

(A) and (B) titration of 15N-labelled N-peptide (residues 363-414; S389E) with wild-type, F209A/D235A and W265G/M287V NiP-CTD. (A) shows the chemical shift difference at 1:0.5 of N-peptide to NiP-CTD for wild-type (black), F209A/D235A (light grey) and W265G/M287V (dark grey). (B) Examples of single-site saturation binding curves fitted to the change in average ¹H and ¹⁵N chemical shifts of Gly385 (squares) and Asp388 (circles) and following titration of ¹⁵N-labelled N-peptide with 8 equivalents of wild-type (top) and F209A/D235A NiP-CTD (middle) or 4 equivalents of W265G/M287V NiP-CTD (bottom) (the latter titration was limited due to solubility W265G/M287V). KD derived from these experiments: 88±4 μM (WT); 122±11 μM (F209A/D235A) and 249±29 μM (W265G/M287V). (C) Luciferase reporter minigenome assay for function of GFP-fused WT, FD or WM Ni—P, or an empty pUC vector in transfected HEK-293T-cells (mean normalised luciferase activity±SD, n=3). (D) IFN-induction luciferase reporter assay using HEK-293-T cells transfected to express the indicated GFP-fused P protein with or without RIG-I-flag or pUC to normalise total transfected DNA (relative luciferase activity±SD, n=3; **, p<0.001, Student's t-test).

FIG. 4: Amino acid substitutions in the STAT1 interacting surface of the P-protein: NMR characterization of F209A/D235A and W265G/M287V NiP-CTD shows minimal structural changes for F209A/D235A NiP-CTD.

(a) Comparison of secondary structure assessed by 13Cαβ chemical shifts of WT (circles), F209A/D235A (squares) and W265G/M287V (triangles) NiP-CTD. Deviation of the measured 13Cα/13Cβ chemical shifts from random coil were measured for each variant. The positive and negative deviations are indicative of the presence of α-helix and β-strand respectively. A schematic representation of the expected secondary structure of NiP-CTD based on the structure of the CTD of P protein from CVS (pdb: 1vyi) is shown. (b) Measurement of the average chemical shift difference (¹H, ¹⁵N) between wild-type and W265G/M287V and (c) between wild-type and F209A/D235A. Differences>0.1 ppm are mapped onto the structure of the CTD of P protein from CVS coloured as dark grey (W265G/M287V) and light grey (F209A/D235A). Sites of mutations are indicated by arrows. The chemical shift difference for the W265G site is 2.4 ppm and is not shown in the plot (b).

FIG. 5: Amino acid substitutions in the STAT1 interacting surface of the P-protein abolish STAT1 antagonist function.

(A and B) Initial data from assays testing the effect of a panel of mutations of P protein for capacity to inhibit P protein-mediated antagonism of IFN signalling, identifying F209A/D235A (A) and A206E/D235K (B) as highly inhibitory. IFN signalling was measured using a dual luciferase assay: HEK-293-T cells were transfected to express the indicated Ni—P proteins (wt wild-type; single and double mutants), or controls (CVS-D30 (or Δ30), CVS-wt), together with pISRE-Luc and pRL-TK plasmids. To activate the IFN signalling pathway, cells were treated without or with IFNα (16 h) before measurement of firefly and Renilla luciferase activity; histograms show normalised luciferase activity calculated relative to that obtained for CVSD30+IFN (positive control). (C) Results of multiple screens using an IFN-dependent luciferase reporter assay to assess antagonist function of the indicated P-proteins expressed in HEK293T cells treated without (−) or with (+) IFN. Data are from multiple screens and so normalized luciferase activity is shown as a percentage of the internal positive control for each assay (IFNα-treated P-ΔC30-expressing cells). Data from each individual assay is the mean of triplicates; where multiple assays were performed, n values (in parentheses) indicate the number of separate assays used to calculate mean±SD.

FIG. 6: Expression and purification of STAT1.

Comparison of yields of STAT1 expressed from pGEX6p3 and pGEV2 vectors. SEC traces show full-length STAT1 expressed as a GST fusion and cleaved post-affinity purification (dashed lines); and as a GB1-fusion post-affinity purification and cleavage of the His6-tag (solid lines).

FIG. 7: Secondary structure analysis of GB1-STAT1 constructs by CD spectroscopy.

(A) Domain structure of the fusion GB1-STAT1: N-terminal domain (ND), coiled-coil domain (CCD), DNA-binding domain (DBD), linker (LK), SH2 domain (SH2) and transactivation domain (TAD). (B) CD spectra of GB1-fused to full length STAT1 and STAT1-CCD-DBD. Data were collected using 0.1-0.2 mg/mL of purified proteins at pH 6.8 and 25° C. (C) Percent secondary structure determined using DichroWeb. In parentheses are calculations of the secondary structure based on domains of the crystal structures of STAT1 (pdb: 1 vyl) and GB1 (pdb: 2qmt). The crystal structure of STAT1 lacks the final C-terminal 57 residues, and hence the values reflect residues 1-683.

FIG. 8: Sedimentation Velocity-Analytical Ultracentifguation (SV-AUC) analysis of STAT1 and its interaction with P protein (GFP—NiP-CTD).

The continuous sedimentation coefficient distributions calculated for (a) GB1-STAT1 and (b) GB1-STAT1-CCD-DBD monitored at 280 nm. GB-STAT1 sediments with a major species at ˜6.5 S (GB1-STAT1-dimer) and with minor species at ˜3.7 (monomer) and at ˜9.5 S (multimer); GB1-STAT1-CCD-DBD sediments at ˜2.9 S (monomeric form). FDS-AUC of 10 μM GFP—NiP-CTD with different concentrations of (c) GB1-STAT1 and (d) STAT1-CCD-DBD. GB1-STAT1 forms a complex with GFP—NiP-CTD at ˜7.2 S, distinct from GFP—NiP-CTD at ˜2.9 S (Inset in c); STAT1-CCD-DBD forms a complex with GFP—NiP-CTD at ˜4 S. AUC experiments were conducted at 50,000 rpm at 20° C.

FIG. 9: Sedimentation velocity of GB1-STAT1.

Upper panels: Radial scans (solid circles) monitored by UV absorbance at 30 μM, 50 μM and 80 μM respectively, with the best-fits to the c(s) sedimentation model overlaid (solid lines). Middle panels: Residuals to the fits. Lower panels: Size distribution plots calculated by best fits of the radial scans to the c(s) sedimentation model.

FIG. 10: Sedimentation velocity of GB1-STAT1-CCD-DBD.

Upper panels: Radial scans monitored by UV absorbance (solid circles) at 10 and 20 μM, with the best-fits to the c(s) sedimentation model overlaid (solid lines). Middle panels: Residuals to the fits. Lower panels: Size distribution plots calculated by best fits of the radial scans to the c(s) sedimentation model.

FIG. 11: Sedimentation velocity of GB1-STAT1 in complex with GFP—NiP-CTD.

Upper panels: Radial scans monitored by fluorescence (solid circles) with 10 μM GFP—NiP-CTD and varying the concentration of GB1-STAT1 (5 to 20 μM), with the best-fits to the c(s) sedimentation model overlaid (solid lines). Middle panels: Residuals to the fits. Lower panels: Size distribution plots calculated by best fits of the radial scans to the c(s) sedimentation model.

FIG. 12: Sedimentation velocity of GB1-STAT1-CCD-DBD in complex with GFP—NiP-CTD.

Upper panels: Radial scans monitored by fluorescence (solid circles) with 5 μM GFP—NiP-CTD and varying the concentration of GB1-STAT1-CCD-DBD (5 to 40 μM), with the best-fits to the c(s) sedimentation model overlaid (solid lines). Middle panels: Residuals to the fits. Lower panels: Size distribution plots calculated by best fits of the radial scans to the c(s) sedimentation model.

FIG. 13: Characterisation of a signal transducer and activator of transcription 1 (STAT1) interacting surface of the P-protein.

Intensities of 2D ¹⁵N, ¹H TROSY cross-peaks from uniformly labelled ²H-¹⁵N NiP-CTD in the presence of full-length GB1-STAT1 (a) without irradiation (−50 ppm) and (b) with irradiation (0.9 ppm). Intensities for resonances of NiP-CTD in the absence of GB1-STAT1 (c) without irradiation (−50 ppm) and (d) with irradiation (0.9 ppm). Peaks indicated by dark grey and light grey boxes are intensities that are reduced >0.5 and within the range 0.6-0.5, respectively, in the presence of GB1-STAT1. The inset in (a) and (b) shows the indole NH of Trp265 which is not attenuated.

FIG. 14: Amino acid substitutions in the STAT1-interacting surface of the P-protein interferes with the interaction of the P-protein with STAT1.

(a) wild-type, (b) W265G/M287V and (c) F209A/D235A-mutated ¹⁵N-labelled NiP-CTD protein (30 μM) were titrated with an equimolar concentration of purified GB1-STAT1; intensity differences are shown in histograms (left panels). Portions of the 2D ¹H-¹⁵N HSQC experiments for the various NiP-CTD proteins with (multiple contours in grey) and without (single contour in black) 30 μM of GB1-STAT1 are shown in the right panels. Spectra are plotted at the same levels and were collected at pH 6.8 and 25° C.

FIG. 15: Structural analysis of P proteins with amino acid substitutions in the STAT1 interacting surface of the P-protein or the W hole of the P-protein.

Wild-type and F209A/D235A NiP-CTD, but not W265G/M287V, show two-state unfolding. (a) Circular Dichroism (CD) spectra of WT, F209A/D235A and W265G/M287V NiP-CTD acquired on 0.1 to 0.2 mg/mL of protein at pH 6.8 and 25° C. The spectra of WT and F209A/D235A are similar, while W265G/M287V shows significantly weaker minima. Secondary structure analysis shows WT 59% helix, 20% strand; F209A/D235A 49% helix, 26% strand and W265G/M287V 44% helix, 29% strand. Thermal stability of (b) WT, (c) W265G/M287V, (d) F209A/D235A NiP-CTD was assessed by CD at 222 nm using 0.1-0.2 mg/ml of proteins dissolved in 50 mM sodium phosphate, 100 mM NaCl, 1 mM DTT, pH 6.8.

FIG. 16: Characterisation of P proteins with amino acid substitutions in the STAT1 interacting surface: NMR characterization of A206E/D235K NiP-CTD.

(a) Measurement of the average chemical shift difference (¹H, ¹⁵N) between wild-type and A206E/D235K. Differences>0.1 ppm are mapped onto the structure of the CTD of P protein from CVS.

FIG. 17: Thermal stability of P protein with amino acid substitutions in the STAT1 interacting surface.

Thermal stability of A206E/D235K NiP-CTD was assessed by CD at 222 nm using 0.1-0.2 mg/ml of proteins dissolved in 50 mM sodium phosphate, 100 mM NaCl, 1 mM DTT, pH 6.8 (compare to Tₘ of 57° C. for WT).

FIG. 18: P protein with amino acid substitutions in the STAT1 interacting surface is deficient for binding to activated STAT1 and prevention of STAT1-DNA binding.

(A) COS-7 cells expressing the indicated GFP-fused WT or mutant Ni—P proteins (FD, F209A/D235A; WM, W265G/M287V) or CVS-PΔ30 were treated with IFNα for the indicated times before lysis for co-IP using GFP-Trap. Lysates and co-IP samples were analysed by immunoblotting (IB) using the indicated antibodies. (B) A DNA fragment containing GAS sequences was incubated without (well 5) or with WT, FD or WM P-CTD (wells 3-4), or with phosphorylated (pY) STAT1 pre-incubated without (wells 6-8) or with the indicated P-CTD (wells 9-20) before gel electrophoresis. Amounts of protein are indicated above gel; Lane 1, 2-log DNA ladder; arrest of the DNA fragment within wells is highlighted by being enclosed in a box.

### DETAILED DESCRIPTION

The present inventors have characterised a signal transducer and activator of transcription 1 (STAT1) interacting surface of the lyssavirus P-protein.

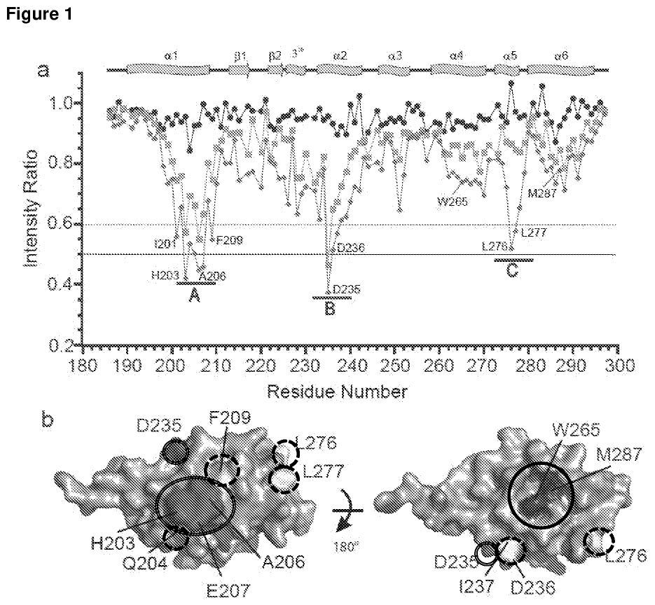

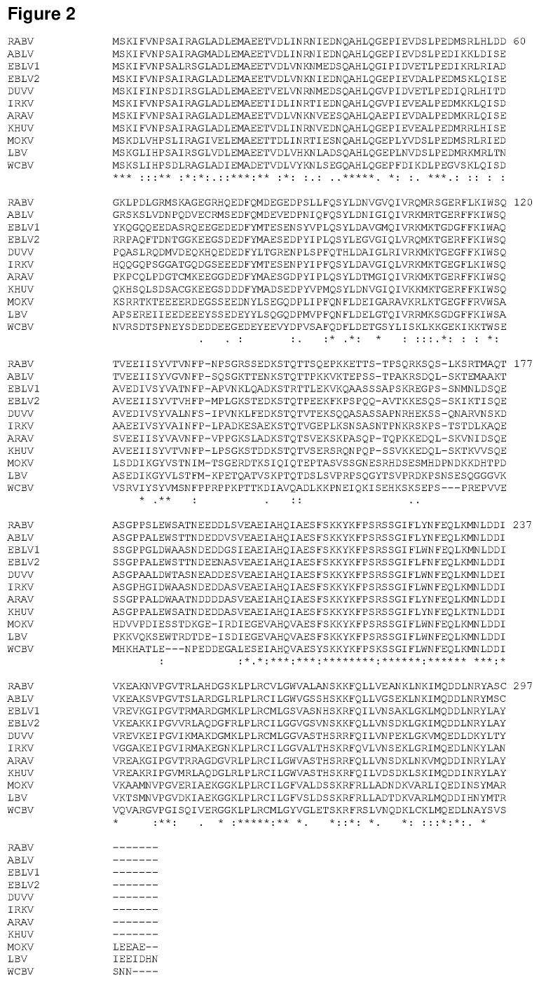

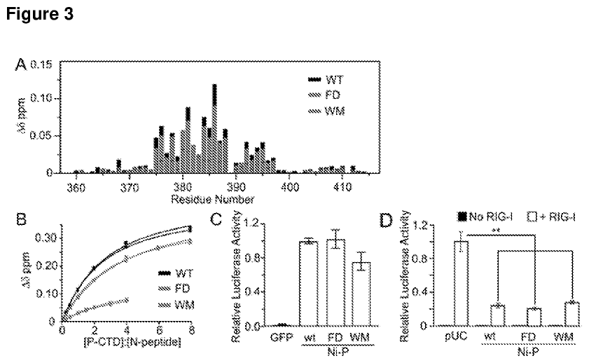

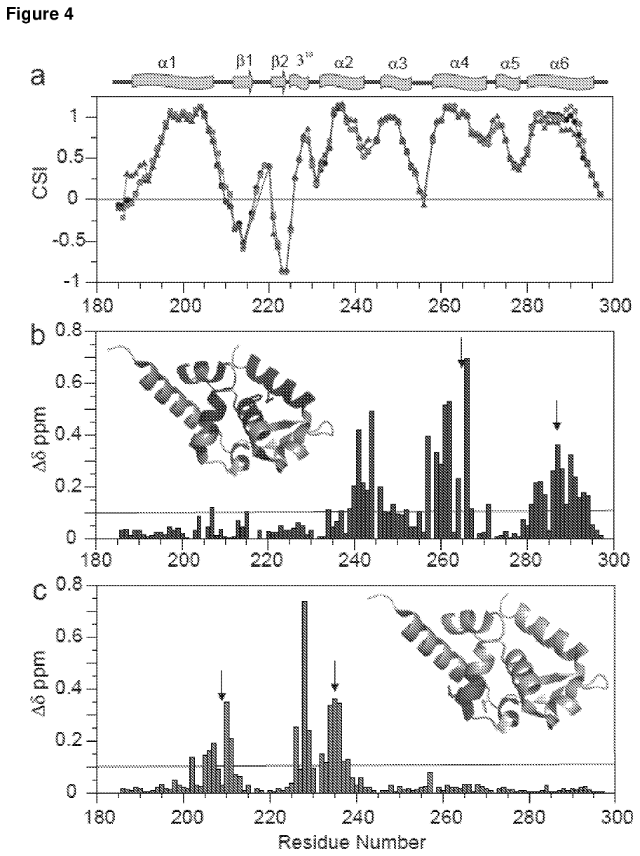

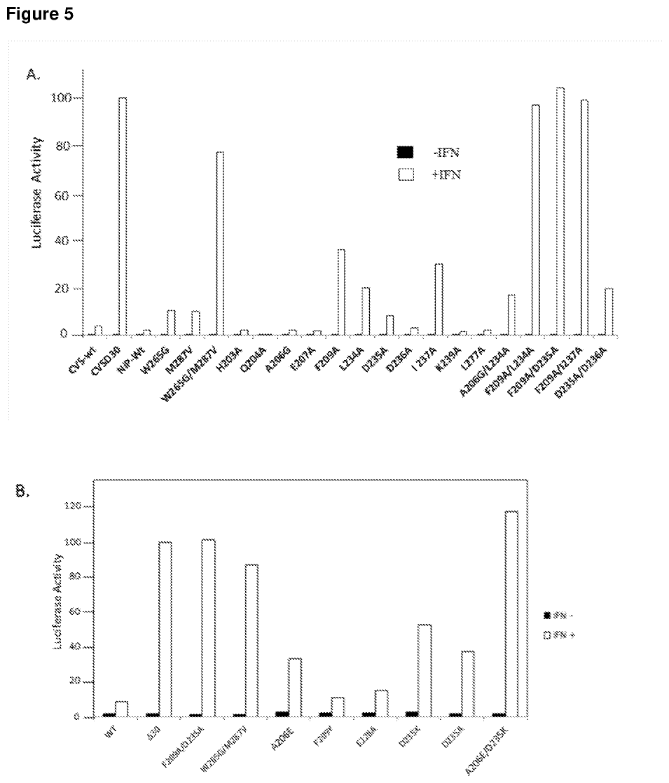

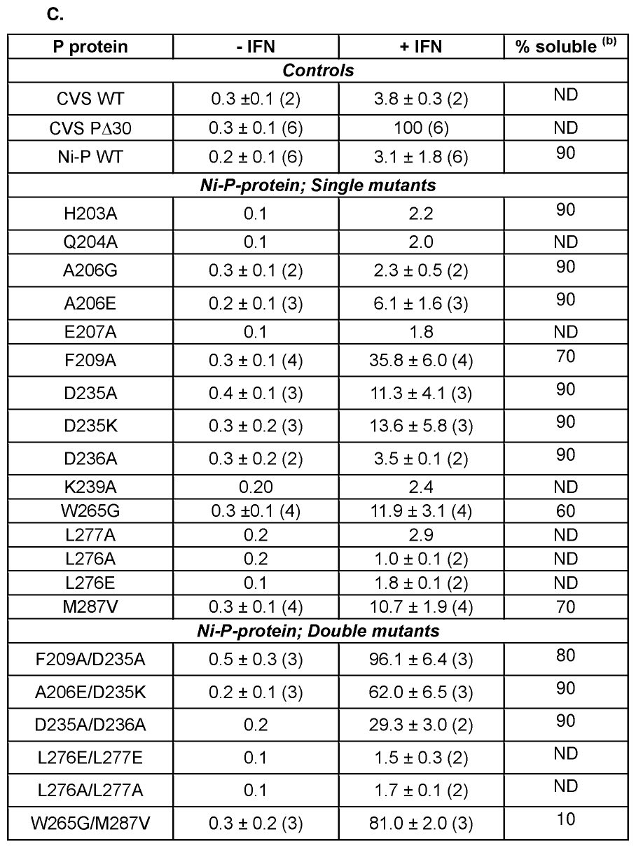

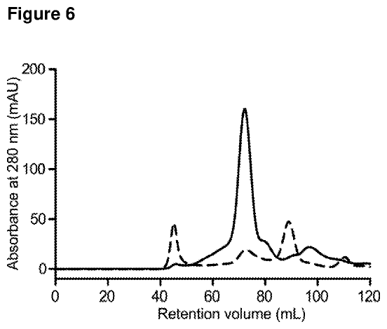

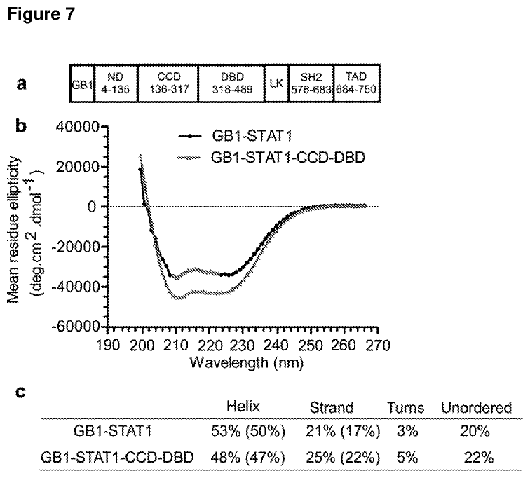

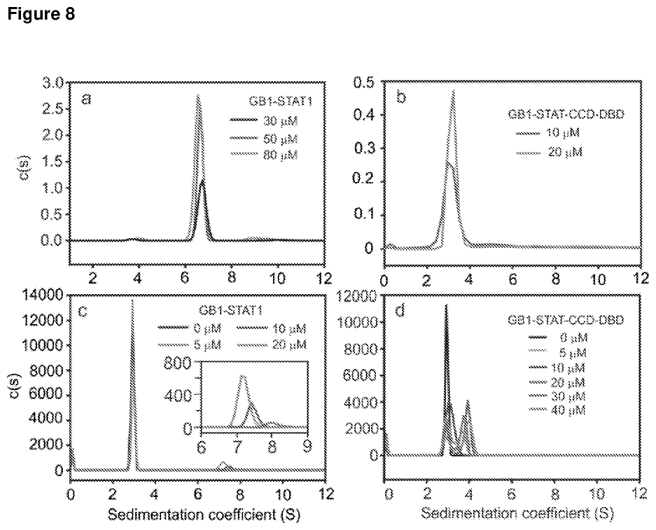

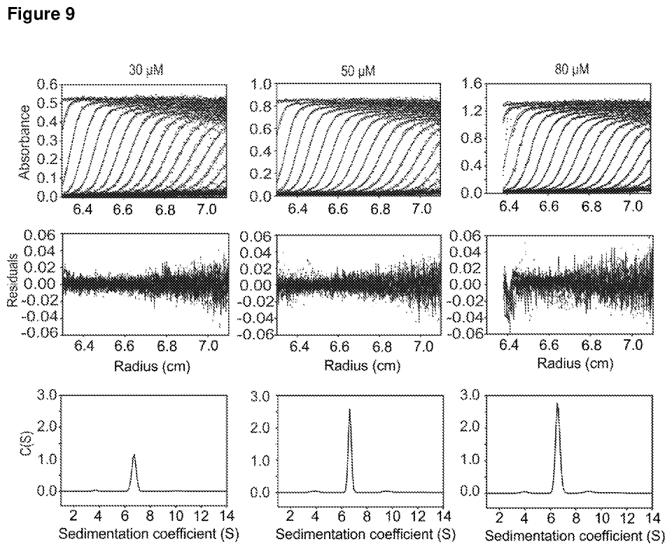

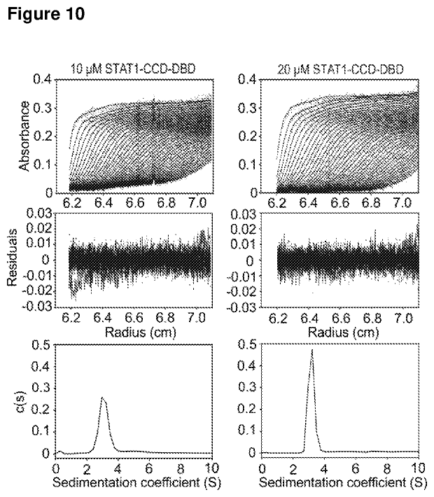

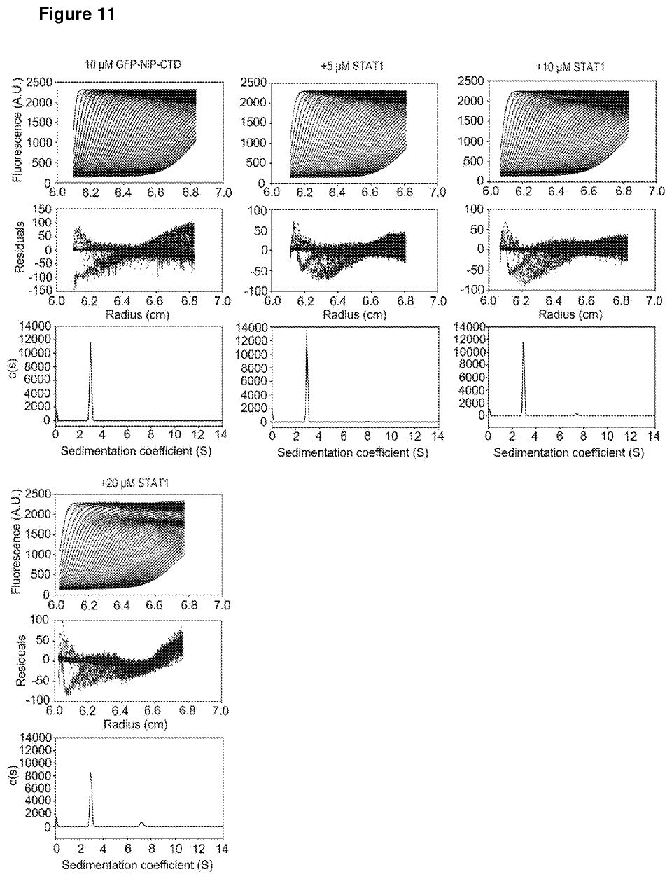

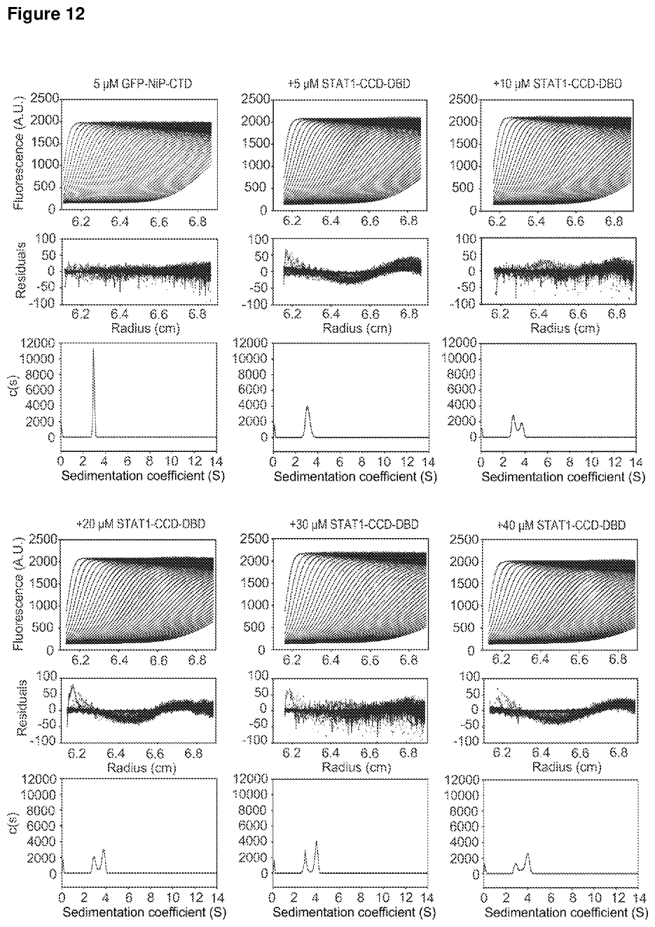

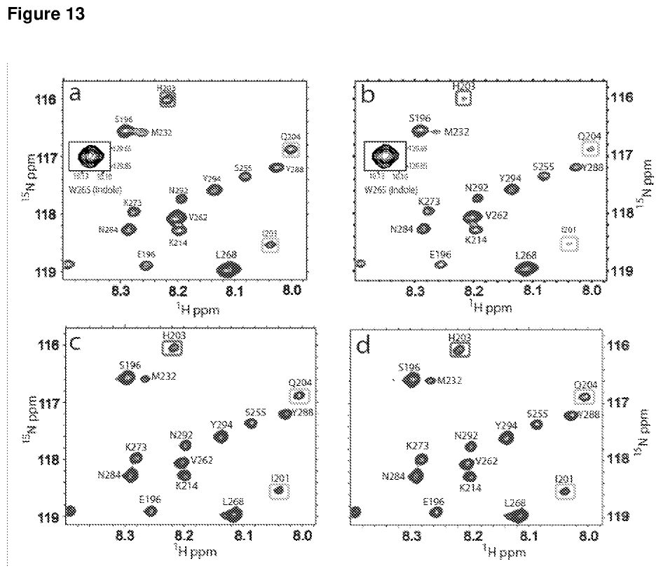

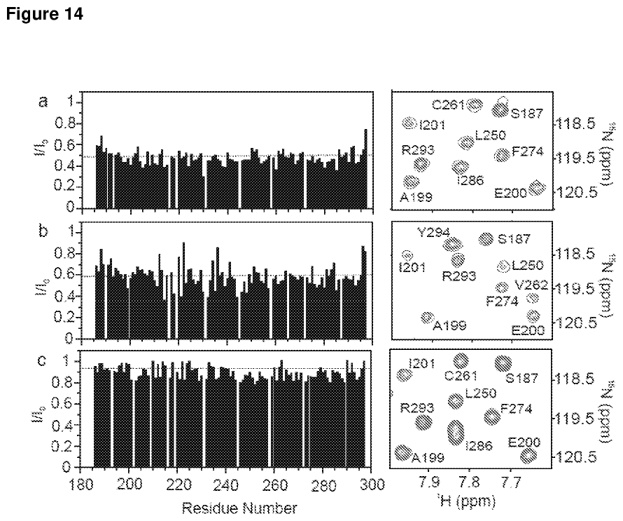

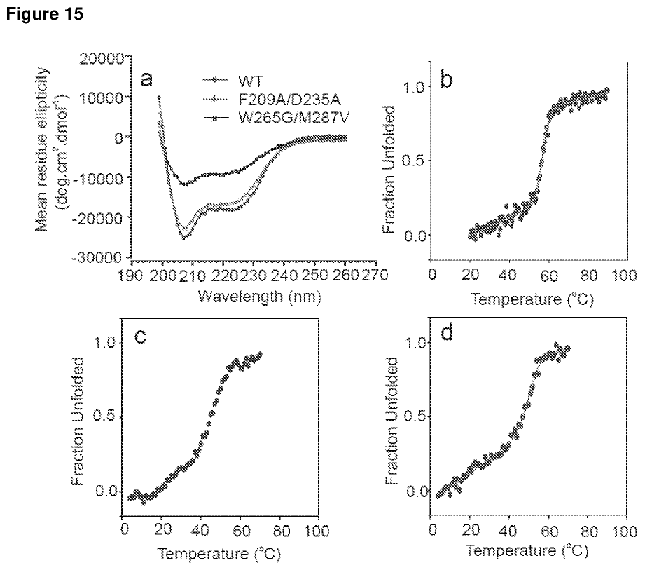

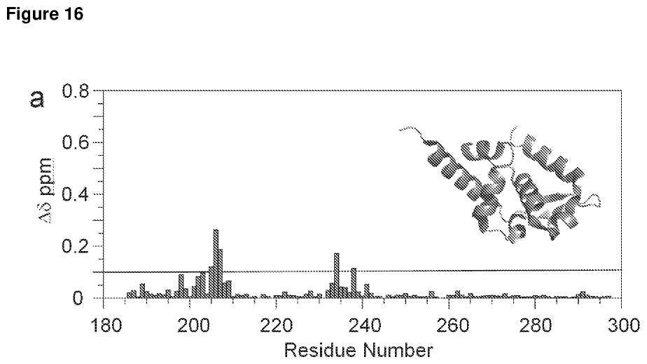

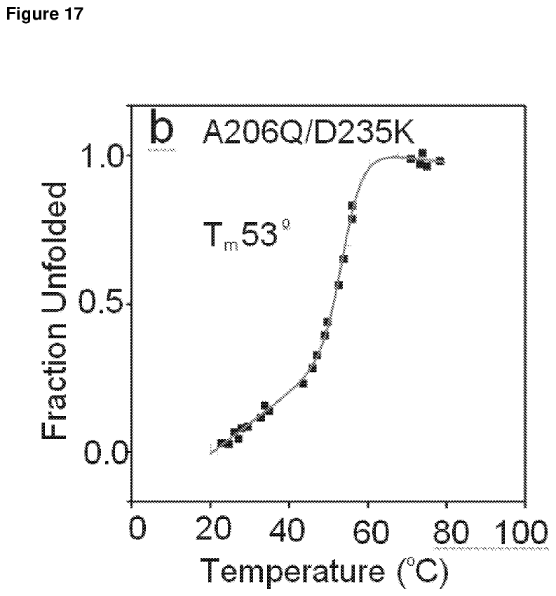

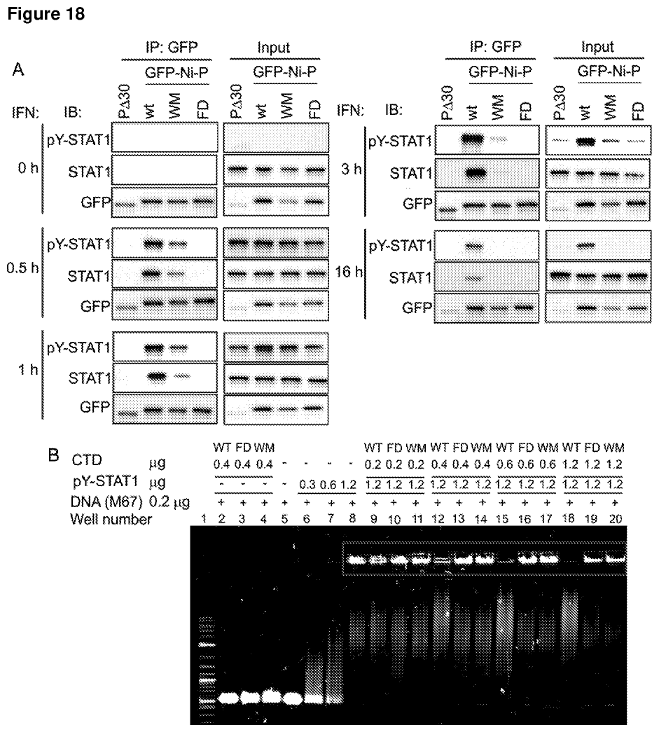

The present inventors have used transferred cross-saturation NMR experiments using truncated and full-length STAT1 to show that P protein (e.g. “NiP-CTD”; where CTD is the C-terminal Domain of the P protein, and Ni refers to the strain Nishigahara) makes contact with multiple domains of STAT1. The STAT1 interacting surface of the lyssavirus P-protein is mostly localized to the round face of the NiP-CTD (FIG. 1b) and is near, but not overlapping, the predicted N-protein binding site. Importantly, this interface is in contrast to the small cleft, the W-hole, previously implicated by mutagenesis as the potential STAT1-binding site. Accordingly, the STAT1 interacting surface excludes the W hole. As described herein, the W-hole corresponds broadly to residues equivalent to L244, P245, C261, W265, and M287 of SEQ ID NO: 1 or equivalent to at least C262, F266, and 1288 of SEQ ID NO 9. At least Trp265 and Met287 of SEQ ID NO: 1 are in the W-hole.

The type-I interferon (IFN) system comprises the earliest immune response of host cells against viral infection. Following detection of infection, host cells release IFNs which bind to the type-I IFN-receptor to activate signaling by members of the signal transducers and activators of transcription (STAT) family, STAT1 and STAT2. This results in entry of STATs to the nucleus and transcriptional activation of hundreds of IFN-stimulated genes (ISGs), which include genes encoding antiviral and immunomodulatory proteins, to establish an antiviral state and facilitate development of the adaptive response.

In resting cells, STATs are generally unphosphorylated (U-STAT) antiparallel dimers, but following receptor engagement, Janus kinases phosphorylate conserved tyrosines in STAT1/2 (pY-STAT1/2), resulting in the formation of parallel dimers that traffic into the nucleus to activate ISGs. The major mediators of type-I IFN signaling are pY-STAT1/2, although pY-STAT1 homodimers also contribute to activate overlapping and distinct ISG subsets. In the nucleus, pY-STAT1/2 heterodimers, with IFN-regulatory factor 9 (IRF9), form the IFN-stimulated gene factor 3 (ISGF3), which binds to IFN-stimulated response elements (ISREs) within gene promoters to stimulate ISG expression. Unphosphorylated STATs (U-STATs) are also known to form functional complexes and mediate transcription of genes relevant to processes including immunity, cell proliferation, and cancer.

Viruses have evolved numerous strategies to overcome the IFN response, mediated principally by viral IFN-antagonist proteins. Given the central role of STATs to responses to IFNs and other cytokines, it is not surprising that many IFN-antagonists interfere with STATs, especially STAT1, by mechanisms including proteasomal degradation, dephosphorylation or inhibition of phosphorylation or nuclear trafficking of STATs. Due to the importance of the IFN response in controlling infection, it has been widely assumed that mechanisms of IFN antagonism play significant roles in pathogenesis. However, studies examining pathogenesis of recombinant viruses defective in specific mechanisms of IFN antagonism are limited.

P-protein is the main IFN antagonist of lyssaviruses, which comprise a genus of highly pathogenic viruses that include RABV and cause rabies disease with a c. 100% case fatality rate (c. 60,000 human deaths/year). In common with many other IFN antagonists, P protein targets multiple stages of the IFN response, including induction and signaling. The latter involves direct interaction with STAT1, which causes STAT1 mislocalisation out of the nucleus via nuclear export and cytoskeletal association of P protein isoforms. Using recombinant rabies viruses containing mutations in P protein, these mechanisms of STAT1 antagonism have been correlated with pathogenicity. P-protein is also an essential co-factor in replication via interaction with nucleocapsid (N) protein associated with genomic RNA (N-RNA), and the viral polymerase (L protein), functions conserved across P gene products of the order Mononegavirales. Thus, P protein is a complex multifunctional protein.

Accordingly, in one embodiment the present invention provides an isolated lyssavirus phosphoprotein (P-protein) comprising one or more amino acid substitutions in a STAT1 interacting surface of the P-protein.

As used herein, the term “P protein” or “Phosphoprotein” or “P protein polypeptide” refers to a polypeptide or protein having all or part of an amino acid sequence of a P protein polypeptide, encoded by a P gene. The term can include regions or fragments of a P proteins. The term includes any phosphoprotein from any member of the lyssavirus genus of RNA viruses.

Exemplary P protein sequences are shown in FIG. 2.

The amino acid sequence of the P protein of Rabies virus (strain Nishigahara RCEH) (RABV) (UNIPROT ACCESSION NUMBER:Q9IPJ8) (SEQ ID NO: 1) is shown below:

MSKIFVNPSAIRAGLADLEMAEETVDLINRNIEDNQAHLQGEPIEVDSLP
EDMSRLHLDDGKLPDLGRMSKAGEGRHQEDFQMDEGEDPSLLFQSYLDNV
GVQIVRQMRSGERFLKIWSQTVEEIISYVTVNFPNPSGRSSEDKSTQTTS
QEPKKETTSTPSQRKSQSLKSRTMAQTASGPPSLEWSATNEEDDLSVEAE
IAHQIAESFSKKYKFPSRSSGIFLYNFEQLKMNLDDIVKEAKNVPGVTRL
AHDGSKLPLRCVLGWVALANSKKFQLLVEANKLNKIMQDDLNRYASC

The amino acid sequence of the P protein of Australian bat lyssavirus (isolate Human/AUS/1998) (ABLV) (UNIPROT ACCESSION NUMBER:Q8JTH2) (SEQ ID NO: 2) is shown below:

MSKIFVNPSAIRAGMADLEMAEETVDLINRNIEDNQAHLQGEPIEVDSLP
EDIKKLDISEGRSKSLVDNPQDVECRMSEDFQMDEVEDPNIQFQSYLDNI
GIQIVRKMRTGERFFKIWSQTVEEIISYVGVNFPSQSGKTTENKSTQTTP
KKVKTEPSSTPAKRSDQLSKTEMAAKTASGPPALEWSTTNDEDDVSVEAE
IAHQIAESFSKKYKFPSRSSGIFLYNFEQLKMNLDDIVKEAKSVPGVTSL
ARDGLRLPLRCILGWVGSSHSKKFQLLVGSEKLNKIMQDDLNRYMSC

The amino acid sequence of the P protein of European bat lyssavirus 1 (strain Bat/Germany/RV9/1968)(EBLV1) (UNIPROT ACCESSION NUMBER:A4UHP9), (SEQ ID NO: 3) is shown below:

MSKIFVNPSALRSGLADLEMAEETVDLVNKNMEDSQAHLQGIPIDVETLP
EDIKRLRIADYKQGQQEEDASRQEEGEDEDFYMTESENSYVPLQSYLDAV
GMQIVRKMKTGDGFFKIWAQAVEDIVSYVATNFPAPVNKLQADKSTRTTL
EKVKQAASSSAPSKREGPSSNMNLDSQESSGPPGLDWAASNDEDDGSIEA
EIAHQIAESFSKKYKFPSRSSGIFLWNFEQLKMNLDDIVREVKGIPGVTR
MARDGMKLPLRCMLGSVASNHSKRFQILVNSAKLGKLMQDDLNRYLAY

The amino acid sequence of the P protein of European bat lyssavirus 2 (strain Human/Scotland/RV1333/2002) (EBLV2) (UNIPROT ACCESSION NUMBER:A4UHQ4) (SEQ ID NO: 4) is shown below:

MSKIFVNPSAIRAGLADLEMAEETVDLVNKNIEDNQAHLQGEPIEVDALP
EDMSKLQISERRPAQFIDNIGGKEEGSDEDFYMAESEDPYIPLQSYLEGV
GIQLVRQMKTGERFFKIWSQAVEEIISYVTVHFPMPLGKSTEDKSTQTPE
EKFKPSPQQAVTKKESQSSKIKTISQESSGPPALEWSTTNDEENASVEAE
IAHQIAESFSKKYKFPSRSSGIFLFNFEQLKMNLDDIVKEAKKIPGVVRL
AQDGFRLPLRCILGGVGSVNSKKFQLLVNSDKLGKIMQDDLNRYLAY

The amino acid sequence of the P protein of Duvenhage virus (DUVV) (UNIPROT ACCESSION NUMBER:056774) (SEQ ID NO: 5) is shown below:

MSKIFINPSDIRSGLADLEMAEETVELVNRNMEDSQAHLQGVPIDVETLP
EDIQRLHITDPQASLRQDMVDEQKHQEDEDFYLTGRENPLSPFQTHLDAI
GLRIVRKMKTGEGFFKIWSQAVEDIVSYVALNFSIPVNKLFEDKSTQTVT
EKSQQASASSAPNRHEKSSQNARVNSKDASGPAALDWTASNEADDESVEA
EIAHQIAESFSKKYKFPSRSSGIFLWNFEQLKMNLDEIVREVKEIPGVIK
MAKDGMKLPLRCMLGGVASTHSRRFQILVNPEKLGKVMQEDLDKYLTY

The amino acid sequence of the P protein of Irkut virus (IRKV) (UNIPROT ACCESSION NUMBER:Q5VKP5) (SEQ ID NO: 6) is shown below:

MSKIFVNPSAIRAGLADLEMAEETIDLINRTIEDNQAHLQGVPIEVEALP
EDMKKLQISDHQQGQPSGGATGQDGSEEEDFYMTESENPYIPFQSYLDAV
GIQLVRKMKTGEGFLKIWSQAAEEIVSYVAINFPLPADKESAEKSTQTVG
EPLKSNSASNTPNKRSKPSTSTDLKAQEASGPHGIDWAASNDEDDASVEA
EIAHQIAESFSKKYKFPSRSSGIFLWNFEQLKMNLDDIVGGAKEIPGVIR
MAKEGNKLPLRCILGGVALTHSKRFQVLVNSEKLGRIMQEDLNKYLAN

The amino acid sequence of the P protein of Aravan virus (ARAV) (UNIPROT ACCESSION NUMBER:Q6X1 D7) (SEQ ID NO: 7) is shown below:

MSKIFVNPSAIRAGLADLEMAEETVDLVNKNVEESQAHLQAEPIEVDALP
EDMKRLQISEPKPCQLPDGTCMKEEGGDEDFYMAESGDPYIPLQSYLDTM
GIQIVRKMKTGERFFKIWSQSVEEIISYVAVNFPVPPGKSLADKSTQTSV
EKSKPASQPTQPKKEDQLSKVNIDSQESSGPPALDWAATNDDDDASVEAE
IAHQIAESFSKKYKFPSRSSGIFLYNFEQLKMNLDDIVREAKGIPGVTRR
AGDGVRLPLRCILGWVASTHSRRFQLLVNSDKLNKVMQDDINRYLAY

The amino acid sequence of the P protein of Khujand virus (KHUV) (UNIPROT ACCESSION NUMBER:Q6X1 D3) (SEQ ID NO: 8) is shown below:

MSKIFVNPSAIRAGLADLEMAEETVDLINRNVEDNQAHLQGEPIEVEALP
EDMRRLHISEQKHSQLSDSACGKEEGSDDDFYMADSEDPYVPMQSYLDNV
GIQIVKKMKTGERFFKIWSQAVEEIISYVTVNFPLPSGKSTDDKSTQTVS
ERSRQNPQPSSVKKEDQLSKTKVVSQEASGPPALEWSATNDEDDASVEAE
IAHQIAESFSKKYKFPSRSSGIFLYNFEQLKTNLDDIVREAKRIPGVMRL
AQDGLRLPLRCILGWVASTHSKRFQILVDSDKLSKIMQDDINRYLAY

The amino acid sequence of the P protein of Mokola virus (MOKV) (UNIPROT ACCESSION NUMBER:P0C569) (SEQ ID NO: 9) is shown below:

MSKDLVHPSLIRAGIVELEMAEETTDLINRTIESNQAHLQGEPLYVDSLP
EDMSRLRIEDKSRRTKTEEEERDEGSSEEDNYLSEGQDPLIPFQNFLDEI
GARAVKRLKTGEGFFRVWSALSDDIKGYVSTNIMTSGERDTKSIQIQTEP
TASVSSGNESRHDSESMHDPNDKKDHTPDHDVVPDIESSTDKGEIRDIEG
EVAHQVAESFSKKYKFPSRSSGIFLWNFEQLKMNLDDIVKAAMNVPGVER
IAEKGGKLPLRCILGFVALDSSKRFRLLADNDKVARLIQEDINSYMARLE
EAE

The amino acid sequence of the P protein of Lagos bat virus (LBV) (UNIPROT ACCESSION NUMBER:056773) (SEQ ID NO: 10) is shown below:

MSKGLIHPSAIRSGLVDLEMAEETVDLVHKNLADSQAHLQGEPLNVDSLP
EDMRKMRLTNAPSEREIIEEDEEEYSSEDEYYLSQGQDPMVPFQNFLDEL
GTQIVRRMKSGDGFFKIWSAASEDIKGYVLSTFMKPETQATVSKPTQTDS
LSVPRPSQGYTSVPRDKPSNSESQGGGVKPKKVQKSEWTRDTDEISDIEG
EVAHQVAESFSKKYKFPSRSSGIFLWNFEQLKMNLDDIVKTSMNVPGVDK
IAEKGGKLPLRCILGFVSLDSSKRFRLLADTDKVARLMQDDIHNYMTRIE
ElDHN

The amino acid sequence of the P protein of Caucasian bat virus (WBCV) (UNIPROT ACCESSION NUMBER:Q5VKP1) (SEQ ID NO: 11) is shown below:

MSKSLIHPSDLRAGLADIEMADETVDLVYKNLSEGQAHLQGEPFDIKDLP
EGVSKLQISDNVRSDTSPNEYSDEDDEEGEDEYEEVYDPVSAFQDFLDET
GSYLISKLKKGEKIKKTWSEVSRVIYSYVMSNFPPRPPKPTTKDIAVQAD
LKKPNEIQKISEHKSKSEPSPREPVVEMHKHATLENPEDDEGALESEIAH
QVAESYSKKYKFPSKSSGIFLWNFEQLKMNLDDIVQVARGVPGISQIVER
GGKLPLRCMLGYVGLETSKRFRSLVNQDKLCKLMQEDLNAYSVSSNN

In one embodiment, the substitutions are relative to a reference P protein sequence, such as those exemplified above. The present inventors have demonstrated that changing a reference or ‘wild-type’ P-protein sequence by substituting at least one amino acid residue by changing the sequence of a polynucleotide encoding the polypeptide can result in the phenotypes described herein.

The present inventors have also demonstrated that, surprisingly, the STAT1 interacting surface comprises amino acid residues that are not strictly conserved across all wild-type lyssaviruses (e.g. FIG. 2 and data not shown), and that surprisingly, substituting such non-conserved amino acids can result in the phenotypes described herein. For example, as described herein (e.g. as shown in FIG. 2), amino acid residues corresponding to amino acid residues 209, 236, 239 of SEQ ID NO: 1 can vary in reference P protein sequences, such as wild type isolates (e.g. including in the reference sequences described herein).

Importantly, the present inventors have also demonstrated that the substitutions made to some residues can indirectly affect the conformation of the STAT1 interacting surface, for example due to a global destabilizing effect on the structure of the NiP-CTD, compromising global protein structure and function, whereas other substitutions can directly affect the STAT1 interacting function of the STAT1 interacting surface, without having a global destabilising effect on protein structure and function (e.g. as measured by solubility). Accordingly, the present invention provides amino acid substitutions that can be made in residues equivalent to those described herein in P-proteins of different isolates, without having a global destabilising effect on protein structure and function of those P-proteins and lyssaviruses encoding those P-proteins.

For example, the present inventors have demonstrated herein that amino acid substitutions can be made in regions and residues of a P-protein without significantly changing solubility of the P protein relative to the solubility of the reference P protein without the one or more substitutions.

Accordingly, in one embodiment, the present invention provides a P protein or a lyssavirus encoding a P-protein as described herein, wherein the solubility of the P-protein is not significantly altered relative to the reference P protein without the one or more substitutions.

In another embodiment, the present invention provides a P protein or a lyssavirus encoding a P-protein as described herein, wherein the solubility of the P-protein is reduced by 25% or less, 20% or less, 15% or less, or 10% or less relative to the reference P protein without the one or more substitutions.

As used herein the term “lyssavirus” refers to a genus of RNA viruses in the family Rhabdoviridae. Members of this genus include rabies virus, Lagos bat virus (LBV), Mokola virus (MOKV), Duvenhage virus (DUVV), European bat lyssavirus-1 (EBLV-1), European bat lyssavirus-2 (EBLV-2), Australian bat lyssavirus (ABLV), Aravan virus (ARAV), Khujand virus (KHUV), Irkut virus (IRKV), West Caucasian bat virus (WCBV) and Shimoni bat virus. Preferably, the virus is rabies virus, or a virus reported as causing rabies in humans such as Mokola virus, Duvenhage virus and Australian bat lyssavirus.

As used herein the term “amino acid substitution” includes substituting one amino acid residue in a polypeptide sequence (e.g. a reference P protein sequence such as a wild-type P protein sequence) for a different amino acid residue in a polypeptide sequence. Amino acid substitutions may be made by changing the sequence of a polynucleotide encoding the polypeptide, for example, changing the sequence of a polynucleotide encoding a lyssavirus P-protein. For example, a reference P protein sequence, such as those exemplified above, is altered by substituting one or more amino acid residues by changing the sequence of a polynucleotide encoding the polypeptide. In one embodiment, the reference protein sequence is a wild-type protein sequence. As used herein a wild-type protein sequence is one that occurs in naturally occurring isolates (e.g. strains isolated from animals or subjects) that have not been actively substituted to change one or more phenotypes of the protein and/or lyssavirus, or in laboratory-adapted strains (e.g. the sequence of the protein in the adapted/fixed strain, which has not been subjected to mutagenesis approaches to produce amino acid substitutions). Such changes may be made by changing one or more nucleotides of a given codon.

The present inventors have also demonstrated that relative to P proteins with a single substitution, as is shown in FIG. 5, more than one substitution in the STAT1 interaction surface can surprisingly have a synergistic effect relative to single substitutions. Importantly, the present inventors have also demonstrated that more than one substitution in the STAT1 interaction (e.g. binding) surface can be made without having a global destabilising effect (e.g. as measured by solubility or another method, such as the biophysical characterizations described herein) on protein structure and function of those P-proteins and lyssaviruses encoding those P-proteins. Accordingly, in one embodiment, the proteins described herein comprise one or more amino acid substitutions.

Amino acid substitutions which, in general, are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine; or (e) any residue is substituted for (or by) a residue with a small side-chain, for example, alanyl.

In the context of the current invention, when at least one amino acid substitution is being introduced to the STAT1 interacting surface of the P-protein, it is preferred that an amino acid mutation is introduced by changing at least two nucleotides of the naturally occurring codon. Without wishing to be bound by theory, this helps to achieve a greater safety margin from spontaneous reversion to wildtype when the protein is utilised in a vaccine. Amino acid substitutions can also be introduced via single nucleotide changes.

As used herein the term “STAT1 interacting surface” refers to the region of a P-protein that interacts/binds to STAT1, for example, by non-covalent interactions such as ionic interactions like attraction of opposite charges on amino acids, hydrogen bonds or hydrophobic interactions. A STAT1 interacting surface includes the amino acid residues involved in interaction/binding, for example, by non-covalent interactions such as ionic interactions like attraction of opposite charges on amino acids, hydrogen bonds or hydrophobic interactions, and amino acids adjacent to such amino acids that are in the STAT1 binding face of a P-protein. Methods for determining a STAT1 interacting surface are described herein, for example, in the Examples. Importantly, as demonstrated herein (for example, in Example 3), the STAT1 interacting surface excludes the W hole. As described herein, the W-hole corresponds broadly to residues equivalent to L244, P245, C261, W265, and M287 of SEQ ID NO: 1 or equivalent to at least C262, F266, and 1288 of SEQ ID NO 9. At least Trp265 and Met287 of SEQ ID NO: 1 are in the W-hole. The present inventors have demonstrated that STAT1 binding does not directly involve the W-hole.

Importantly, many residues of the STAT1 interacting surface of the P-protein are highly conserved amongst the lyssavirus genus suggesting a shared mechanism for antagonizing IFN-mediated STAT1 activation, although as is shown in the present application, residues of the STAT1 interacting surface may vary in P-proteins of wild-type isolates. The STAT1 interacting surface of the P-protein characterised using NMR and assays in mammalian cells with or without activation by IFN, indicate that the STAT1 interacting surface of the P-protein is common to unphosphorylated STAT1 (U-STAT1) and phosphorylated (pY)-STAT1. Notably, FIG. 5A shows Ni—P F209A/D235A and A206E/D235K, each comprised of two residues within two main regions of the characterised STAT1 interacting surface of the P-protein, was as potent as the control protein PΔ30 that lacks a structured CTD, confirming the critical role of these residues. FIG. 5C similarly shows the critical role of these residues.

As discussed above, in one aspect the present invention provides an isolated lyssavirus phosphoprotein (P-protein) comprising one or more amino acid substitutions in a signal transducer and activator of transcription 1 (STAT1) interacting surface of the P-protein.

The present inventors have characterised the STAT1 interacting surface, which is within the C-terminal domain (CTD) of the P-protein.

Accordingly, the present invention provides an isolated lyssavirus P-protein as described herein, wherein the STAT1 interacting surface is within the C-terminal domain (CTD) of the P-protein.

In one aspect the present invention therefore provides an isolated lyssavirus P-protein as described herein, wherein the one or more amino acid substitutions is within the C-terminal domain (CTD) of the P-protein.

As used herein the term “C terminal domain” includes the C terminal region of a lyssavirus P-protein, and includes the region corresponding to residues 186 to 297 of SEQ ID NO: 1.

In one aspect the present invention therefore provides an isolated lyssavirus P-protein as described herein, wherein the one or more amino acid substitutions is within the region corresponding to residues 186 to 297 of SEQ ID NO: 1.

The globular C-terminal domain of P protein (P-CTD) contains sites required for binding to STAT1 and N-RNA and so is central to the functions of P protein in replication and immune evasion.

The present inventors have demonstrated that the C-terminal domain contains a number of regions which form part of the STAT1 interacting surface of a P-protein.

As used herein the term “region A” is the region corresponding to residues 200 to 210 of SEQ ID NO: 1. As used herein the term “region B” is the region corresponding to residues 234 to 239 of SEQ ID NO: 1. As used herein the term “region C” is the region corresponding to residues 275 to 278 of SEQ ID NO: 1.

Accordingly, in one aspect the present invention provides an isolated lyssavirus P-protein as described herein, wherein the one or more amino acid substitutions is within, or adjacent to, a helix 1, a helix 2 and/or a helix 5 of the of the C-terminal domain of the P-protein, for example, as shown in FIG. 1.

The present inventors have demonstrated that one or more amino acid substitutions in the STAT1 interacting surface of a P-protein interfere with the interaction of the P-protein with STAT1 and/or modulate IFN antagonistic activity of the P-protein.

In this study the present inventors have used nuclear magnetic resonance (NMR) spectroscopy to elucidate the STAT1 binding interface on P-CTD and show that despite clear effects of W-hole mutations on P protein-STAT1 interaction, the W-hole does not contact STAT1, and several distinct, novel sites within P-CTD that comprise the binding surface have been identified. By using full-length recombinant STAT1 to elucidate the full extent of the interface, the present inventors further show that the P-CTD binding site lies on the DBD and CCD of STAT1, but also appears to involve contact with either the STAT1 N-terminal or C-terminal domains, indicating that virus-STAT complexes can involve a complex interface. The present inventors have also directly validated specific functions in STAT1 targeting of contact residues in P-CTD, mutation of which produces largely localized effects, in contrast to mutations of the W-hole that appear to act via broader structural effects.

Accordingly, in one aspect the present invention provides an isolated lyssavirus P-protein as described herein, wherein the one or more amino acid substitutions interferes with the interaction of the P-protein with STAT1. As discussed above, as used herein, the term “P protein” refers to a polypeptide or protein having all or part of an amino acid sequence of a P protein polypeptide, encoded by a P gene.

As used herein, the terms “Interaction”, “binding” or “specifically interacting”, “specifically binding”, refer to the interaction between binding pairs (e.g. STAT1 and P protein). In general, the terms “Interaction”, “binding” or “specifically interacting”, “specifically binding” refer to the specific interaction of one compound to another, wherein the level of interaction, as measured by any standard assay, including those described herein, is higher than an assigned cut-off value.

Importantly, the present inventors have demonstrated that one or more amino acid substitutions can modulate IFN antagonistic activity of the P-protein. Accordingly, in one aspect the present invention provides an isolated lyssavirus P-protein as described herein, wherein the one or more amino acid substitutions modulates IFN antagonistic activity of the P-protein. P-protein interaction with STAT1 causes nuclear exclusion of P-protein-STAT complexes, and therefore P-protein can inhibit activation of IFN-dependent genes. Accordingly, in one aspect the present invention provides an isolated lyssavirus P-protein as described herein, wherein the one or more amino acid substitutions inhibits the ability of P protein to antagonise activation of IFN-dependent genes.

As used herein, the term “antagonistic activity” refers to the ability of a given P-protein to interact with STAT1, thereby preventing a biological activity of STAT1, such as interferon type I (IFN-alpha and IFN-beta) and type II (IFN-gamma) signaling in the host cell. As used herein the term “modulates” refers to a change in the amount, quality, or effect of a particular response or activity, such as a biological activity of STAT1, (or for example, IFN antagonist activity). Both increases and decreases in the response or activity are included.

In one embodiment, the ability of the substituted P protein or lyssavirus encoding a substituted P protein to antagonise activation of IFN-dependent genes is reduced relative to a wild-type P protein or lyssavirus encoding a wild-type P protein by at least 5, 10, 15, 20, 25, 29, 30, 35, 40, 45, 50, 55, 60, 62, 65, 70, 75, 80, 82, 85, 90, 95, or 96%.

In one aspect the present invention provides isolated lyssavirus P-protein as described herein, wherein the one or more amino acid substitutions are in the region corresponding to amino acid residues 200 to 278 of SEQ ID NO: 1.

As used herein the term “corresponding to” and “equivalent conserved position” refer to an amino acid residue of a first P-protein that corresponds to an amino acid residue at an equivalent position of a second P-protein. For example, as shown in FIG. 2, the sequences of lyssaviruses P-proteins can be aligned to determine conserved and highly conserved residues of lyssaviruses P-proteins, and the positions of the conserved and highly conserved residues can differ in position (e.g. residue number from the N and/or C terminal ends of the P protein). Reference throughout the specification to mutation or substitution of a specific residue in one type of lyssavirus would be understood to encompass mutation or substitution of the corresponding amino acid residue in another lyssavirus, irrespective of whether that that residue may be at a different residue number from the N and/or C terminal ends of the P-protein.

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence, a segment of a polypeptide sequence or a full length polypeptide sequence.

(b) As used herein, “comparison window” makes reference to a contiguous and specified segment of a polynucleotide or polypeptide sequence, wherein the polynucleotide or polypeptide sequence in the comparison window may comprise additions or deletions (i.e., gaps) compared to the reference sequence (which does not comprise additions or deletions) for optimal alignment of the two polynucleotides or polypeptides. Generally, in the case of nucleotides, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide or polypeptide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, as modified in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL W (2.1) in the PC/Gene program (available from Intelligenetics, Mountain View, Calif., and as used herein in FIG. 2); the ALIGN program (Version 2.0) and GAP, BESTFIT, BLAST, FASTA, and TFASTA in the GCG Wis. Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM 120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See the National Center for Biotechnology Information website on the world wide web at www.ncbi.hlm.nih.gov. Alignment may also be performed manually by inspection.

Unless otherwise stated, sequence identity/similarity values provided herein refer to values that can be obtained using CLUSTAL W using the CLUSTAL W multiple sequence alignment algorithm (version 1.83), with the default pairwise alignment parameters, and a scoring method in percentage; or obtained using any equivalent program thereof, such as CLUSTAL (e.g. CLUSTAL W (2.1)), GAP Version 10, MatGAT Vector NTI etc. The term “equivalent program” as used herein refers to any sequence comparison program that, for any two or more sequences in question, generates an alignment having identical nucleotide or amino acid residue matches and an identical percent sequence identity when compared to the corresponding alignment generated by CLUSTAL W.

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

In one aspect, the present invention provides an isolated lyssavirus P-protein as described herein, wherein the one or more amino acid substitutions are at an amino acid residue corresponding to amino acid residues 203, 204, 206, 207, 209, 228, 234, 235, 236, 239 and/or 277 of SEQ ID NO: 1.

In one aspect, the present invention provides an isolated lyssavirus P-protein as described herein, wherein the one or more amino acid substitutions are at an amino acid residue corresponding to amino acid residues 203, 204, 206, 207, 209, 228, 234, 235, 236, 239 and/or 277 of SEQ ID NO: 1, wherein the substitutions are 203A, 206G, 207A, 209A, 209Y, 228A, 234A, 235A, 235K, 236A, 239A, and/or 277A.

In one aspect, the present invention provides an isolated lyssavirus P-protein as described herein, wherein the P protein comprises at least two amino acid substitutions in a signal transducer and activator of transcription 1 (STAT1) interacting surface. For example, in one embodiment the P-protein comprises at least two amino acid substitutions in a STAT1 interacting surface. In another embodiment, the P-protein comprises at least three amino acid substitutions in a STAT1 interacting surface. In another embodiment, the P-protein comprises at least four amino acid substitutions in a STAT1 interacting surface.

In another aspect, the present invention provides an isolated lyssavirus P-protein as described herein, wherein the P-protein further comprises at least one amino acid substitution not in a STAT1 interacting surface.

For example, it is envisaged that additional amino acid substitutions may be included in other parts of the P-protein wherein the amino acid substitutions do not prevent the ability of a virus comprising the P protein to replicate.

In one aspect, the present invention provides an isolated lyssavirus P-protein as described herein, wherein the at least two amino acid substitutions are at amino acid residues selected from the group consisting of amino acid residues corresponding to amino acid residues 206, 209, 235 and 236 of SEQ ID NO: 1.

In one aspect, the present invention provides an isolated lyssavirus P-protein as described herein wherein the two amino acid substitutions are selected from the group consisting of A206E, F209A, D235A, D235K, D236A or an equivalent corresponding position.

In one aspect, the present invention provides an isolated lyssavirus P-protein as described herein, wherein the at least two amino acid substitutions are at amino acid residues corresponding to amino acid residues 209 and 235 of SEQ ID NO: 1, amino acid residues corresponding to amino acid residues 206 and 235 of SEQ ID NO: 1, and/or amino acid residues corresponding to amino acid residues 235 and 236 of SEQ ID NO: 1.

In one aspect, the present invention provides an isolated lyssavirus P-protein as described herein wherein the two amino acid substitutions are selected from F209A and D235A, or A206E and D235K, or D235A and D236A, or an equivalent corresponding position.

NMR titrations (FIG. 3) show that NiP-CTD W265G/M287V retains the ability, albeit more weakly, to bind to STAT1; hence the present inventors propose that these mutations indirectly affect the conformation of the interface due to a global destabilizing effect on the structure of the NiP-CTD.

Accordingly, in one aspect the present invention provides an isolated lyssavirus P-protein as described herein, wherein the P-protein does not comprise an amino acid substitution in the W-hole of the P-protein.

As used herein, the term W hole refers to a hydrophobic cleft, or pocket, of the P-CTD. The W-hole corresponds broadly to residues equivalent to L244, P245, C261, W265, and M287 of SEQ ID NO: 1 or equivalent to at least C262, F266, and 1288 of SEQ ID NO 9. At least Trp265 and Met287 of SEQ ID NO: 1 are in the W-hole.

While assessment of the secondary structure by NMR for NiP-CTD F209A/D235A, A206E/D235K and W265G/M287V compared with wild-type NiP-CTD showed minimal differences (FIGS. 4 and 16), additional biophysical characterization of NiP-CTD W265G/M287V indicated that it is markedly and globally destabilized. The combined mutations W265G and M287V show incomplete loss of binding of STAT1 in NMR assays compared with the F209A/D235A mutation, suggesting that W265G/M287V mutation does not directly affect the binding site, and instead affects STAT1 binding via a conformational effect resulting in a weakened, but not entirely ablated interaction. Consistent with this, W265G/M287V NiP protein was defective in antagonism of STAT1 signalling in mammalian cells compared with wild-type protein, but apparently to a lesser extent than F209/D235A or A206E/D235K NiP protein.

The data described herein indicates that substitutions within the newly identified interaction site (such as F209A/D235A and A206E/D235K) can provide new, minimally disruptive mutations for vaccine attenuation, which can be used either alone or in combination with W-hole mutations or mutations elsewhere in P protein or the viral genome to improve safety of ‘STAT-blind’ viruses.

Accordingly, in one aspect the present invention provides an isolated lyssavirus P-protein as described herein, wherein the P protein further comprises one or more amino acid substitutions in the W-hole of the P-protein. In one aspect the present invention provides an isolated lyssavirus P-protein as described herein, wherein one or more amino acid substitutions are at an amino acid residue corresponding to amino acid residue 265 and/or or 287 of SEQ ID NO: 1. In one aspect the present invention provides an isolated lyssavirus P-protein as described herein, wherein the amino acid substitutions are 265G or 287V of SEQ ID NO: 1, or an equivalent conserved position.

In one embodiment, the substitutions in a lyssavirus protein are relative to a reference protein sequence, such as a wild-type protein sequence.

Accordingly, in some embodiments the lyssavirus protein may naturally comprise one or more preferred amino acid substitutions relative to a reference protein sequence, and therefore no substitution of those one or more preferred amino acid substitutions is required. For example, the DUVV P protein comprises 265G; so to produce a virus expressing a P protein with, for example, 265G and 287V, no substitution is required at amino acid residue 265.

Despite the proximity of the STAT1 interacting surface of the P-protein and N-RNA binding sites in P-CTD the present inventors have shown that mutations can be introduced that specifically impact the former without strong detriment to the latter. For example, the F209A/D235A mutation produced effects on STAT1 interaction and antagonism, with little to no effect on the affinity of the interaction with N protein, demonstrating that critical residues/surfaces required for STAT1 binding and virus replication are separate. Nevertheless, the demonstration of proximity of region A of the STAT1 interacting surface of the P-protein, and the predicted N-binding site suggest that binding to these proteins is tightly coordinated by P protein.

Without wishing to be bound by theory, the amino acid substitutions described herein allow the ability of producing attenuated, viral vaccines without detriment to viral replication, and the safety profile of these vaccines allows them to be deployed in populations of subjects (e.g. non-human animals).

The present inventors have demonstrated that despite ablation of STAT1 antagonist function, replication function of F209A/D235A P-protein was equivalent to that of WT.

Accordingly, in one aspect, the present invention provides an isolated lyssavirus P-protein as described herein, wherein the one or more amino acid substitutions do not significantly alter replication.

Accordingly, in one aspect, the present invention provides an isolated lyssavirus P-protein as described herein, wherein the one or more amino acid substitutions do not abolish polymerase cofactor function.

For example, in a preferred embodiment, the one or more acid substitutions do not abolish viral transcription, and/or abolish viral replication, and/or abolish binding to N protein. In another preferred embodiment the one or more acid substitutions allow viral transcription, and/or allow viral replication, and/or allow binding to N protein.

Accordingly, in one aspect the present invention provides an isolated lyssavirus P-protein as described herein, wherein the one or more amino substitutions are not in the N-protein interacting surface.

In another aspect the present invention provides an isolated lyssavirus P-protein as described herein, wherein the one or more amino substitutions are within in the N-protein interacting surface. In another aspect the present invention provides an isolated lyssavirus P-protein as described herein, wherein the one or more amino substitutions are within in the N-protein interacting surface, wherein the amino acid substitutions do not prevent the ability of a P protein to interact/bind to N protein.

As used herein the term “N protein interacting surface” refers to the region of a P protein that interacts/binds to N protein, for example, by non-covalent interactions such as ionic interactions like attraction of opposite charges on amino acids, hydrogen bonds or hydrophobic interactions. A N protein interacting surface includes the amino acid residues involved in interaction/binding, for example, by non-covalent interactions such as ionic interactions like attraction of opposite charges on amino acids, hydrogen bonds or hydrophobic interactions. The N-protein interacting surface also includes the amino acid residues adjacent to the amino acid residues involved in interaction/binding (e.g. residues that are in the N protein binding face of a P protein). Methods for determining a N protein interacting surface are described herein, for example, in the Examples.

In one aspect the present invention provides an isolated lyssavirus P-protein as described herein, wherein the lyssaviruses is selected from the group consisting of rabies virus, Lagos bat virus (LBV), Mokola virus (MOKV), Duvenhage virus (DUVV), European bat lyssavirus-1 (EBLV-1), European bat lyssavirus-2 (EBLV-2), Australian bat lyssavirus (ABLV), Aravan virus (ARAV), Khujand virus (KHUV), Irkut virus (IRKV), West Caucasian bat virus (WCBV) and Shimoni bat virus. Preferably, the virus is rabies virus, or a virus reported to cause rabies in humans such as Mokola virus, Duvenhage virus and Australian bat lyssavirus.

Importantly, the present inventors have generated nucleic acids encoding the P-proteins characterised herein. Accordingly, in one aspect the present invention provides an isolated nucleic acid encoding a P-protein as described herein, or a complement/antigenome thereof.

Traditional RNA virus vaccines are from naturally attenuated isolates, which are difficult to control and provide unpredictable results. Reverse genetics technology makes it possible to manipulate RNA viruses as DNA, which can be mutated, deleted or reconstructed according to deliberate designs. Reverse genetics involves reverse transcription of the RNA viral genome into cDNA, and cloning into a vector, such as a plasmid. After transfection of host cells, the vector is transcribed into RNA, to be encapsidated by viral proteins, which can also be supplied by plasmids. The encapsidated RNA forms a ribonucleoprotein complex; the ribonucleoprotein complexes can provide templates for expression of viral mRNAs or genome replication, or can be packaged into virions that can be recovered.

An efficient reverse genetics system based on the rabies virus ERA strain is described in PCT Publication No. WO 2007/047459, which is incorporated herein by reference. This rabies reverse genetics system is useful for a variety of purposes, including to attenuate virus in a defined manner for vaccine development and to produce virus vectors for expression of heterologous proteins, such as a protein from another lyssavirus for the generation of a pan-lyssavirus vaccine.

Recombinant viruses with favourable properties for vaccination can be designed using, for example, the reverse genetics system disclosed in PCT Publication No. WO 2007/047459.

Lyssaviruses are composed of two major structural components, a nucleocapsid or ribonucleoprotein (RNP), and an envelope in the form of a bilayer membrane surrounding the RNP core. The infectious component of all Lyssaviruses is the RNP core, which consists of the negative strand RNA genome encapsidated by nucleoprotein (N) in combination with RNA-dependent RNA-polymerase (L) and phosphoprotein (P). The envelope surrounding the RNP contains the trans-membrane glycoprotein (G), and a layer of matrix (M) protein underlies the inner face of the envelope. Thus, the viral genome codes for these five proteins: the three proteins in the RNP (N, L and P), the matrix protein (M), and the glycoprotein (G).

In one aspect, the present invention provides a lyssavirus genome as described herein, wherein the viral genome encodes a protein comprising at least one further amino acid substitution. In one aspect, the viral genome encodes a protein comprising at least one further amino acid substitution in the N, L, P, M and/or G proteins.

For example, the G-protein mediates cell entry and elicits the production of neutralising antibodies, and has been the focus of previous attenuation efforts. In particular, the Arginine residue at position 333 of the G-protein has been shown to contribute to rabies virus pathogenicity as its mutation can attenuate virus (see PCT Publication No. WO 2007/047459, which is incorporated herein by reference). Accordingly, in aspects of the present invention where the viral genome encodes a protein comprising at least one further amino acid substitution, in one embodiment the at least one further amino acid substitution is an amino acid substitution of an amino acid residue corresponding to amino acid residue 333 of the G-protein.

In the context of a virus with a negative-strand RNA genome (such as the genome of a lyssavirus), “antigenome” refers to the complement (positive strand) of the negative strand genome.

A P-protein encoding gene of the invention may be contained within, and expressed as part of, a full-length lyssavirus genome. Accordingly, in another aspect of the invention, there is provided a recombinant lyssavirus genome encoding a phosphoprotein (P-protein) as described herein.

In another aspect the present invention provides a lyssavirus genome, wherein the complement of the lyssavirus genome encodes a P-protein as described herein.

In one aspect, the present invention provides a lyssavirus comprising a lyssavirus genome as described herein. In one aspect, the present invention provides a lyssavirus virion comprising a lyssavirus genome as described herein.

In another aspect, the present invention provides a lyssavirus virion comprising a lyssavirus genome as described herein, wherein the lyssavirus is attenuated.

In another aspect, the present invention provides a lyssavirus virion comprising a lyssavirus genome as described herein, wherein the lyssavirus is able to replicate.

For production of lyssavirus, cell lines such as BHK2.1, Vero and Nil-2 can be used as would be known to the skilled person in the art. Preferably Vero cells or other interferon-deficient cell lines are used as they do not produce interferon, so will not inhibit growth of the STAT-blind virus, thus enabling production of high titre preparations of lyssavirus, and/or large volumes of preparations of lyssavirus.

In one embodiment, the lyssavirus encodes a P-protein comprising one or more amino acid substitutions in a signal transducer and activator of transcription 1 (STAT1) interacting surface of the P-protein.

In one embodiment, the lyssavirus encodes a P-protein wherein the interacting surface is within the C-terminal domain (CTD) of the P-protein.

In one embodiment, the lyssavirus encodes a P-protein, wherein the interacting surface is within the region corresponding to residues 186 to 297 of SEQ ID NO: 1.

In one embodiment, the lyssavirus encodes a P-protein, wherein the one or more amino acid substitutions is within or adjacent to a helix 1, a helix 2 and/or a helix 5 of the of the C-terminal domain of the P-protein.

In one embodiment, the lyssavirus encodes a P-protein, wherein the one or more amino acid substitutions are between a helix 1, a helix 2 and/or a helix 5 of the of the C-terminal domain of the P-protein.

In one embodiment, the lyssavirus encodes a P-protein, wherein the one or more amino acid substitutions interfere with the interaction of the P-protein with STAT1.

In one embodiment, the lyssavirus encodes a P-protein, wherein the P-protein does not comprise an amino acid substitution in the W-hole of the P-protein. In an alternative embodiment, the lyssavirus encodes a P-protein comprising one or more amino acid substitutions in a signal transducer and activator of transcription 1 (STAT1) interacting surface of the P-protein, wherein the P-protein comprises one or more amino acid substitution in the W-hole of the P-protein.

In one embodiment, the lyssavirus encodes a P-protein, wherein the one or more amino acid substitutions do not abolish polymerase cofactor function.

In one embodiment, the lyssavirus encodes a P-protein as described herein, wherein the one or more amino acid substitutions are not in the N-protein interacting surface. In an alternative embodiment, the lyssavirus encodes a P-protein as described herein wherein the one or more amino acid substitutions are in the N-protein interacting surface. In another aspect, the present invention provides a lyssavirus as described herein, wherein the one or more amino acid substitutions are in the N-protein interacting surface, and wherein the amino acid substitutions do not prevent the ability of a P protein to interact/bind to N protein.

In one embodiment, the lyssavirus encodes a P-protein, wherein the one or more amino acid substitutions modulate IFN antagonistic activity of the P-protein.

In one embodiment the lyssaviruses is selected from the group consisting of including rabies virus, Lagos bat virus (LBV), Mokola virus (MOKV), Duvenhage virus (DUVV), European bat lyssavirus-1 (EBLV-1), European bat lyssavirus-2 (EBLV-2), Australian bat lyssavirus (ABLV), Aravan virus (ARAV), Khujand virus (KHUV), Irkut virus (IRKV) and West Caucasian bat virus (WCBV) and Shimoni bat virus. Preferably, the virus is rabies virus, or a virus reported to cause rabies in humans such as Mokola virus, Duvenhage virus and Australian bat lyssavirus.

As used herein the term “attenuated”, in the context of a live virus, such as a lyssavirus virion, refers to the ability of the virion/virus to infect a cell or subject and/or its ability to produce disease being reduced. Typically, an attenuated virus retains at least some capacity to elicit an immune response following administration to an immunocompetent subject. In some cases, an attenuated virus is capable of eliciting a protective immune response without causing any signs or symptoms of infection.

A lyssavirus or lyssavirus virion as described herein can be used in an immune-stimulating composition.

As used herein, the term “immune-stimulating” and “immune stimulation” refer to the generation of an immune response upon administration of a composition to a subject, such as a lyssavirus or lyssavirus virion as described herein. The immune response may be prophylactic or therapeutic.

In one aspect, the immune-stimulating composition is formulated with a pharmaceutically acceptable carrier in order to make it suitable for administration to a subject.

The amino acid substitutions described herein can be used for diverse lyssaviruses (e.g. different lyssavirus genotypes).

The immunogenic compositions provided herein are contemplated for use with both human and non-human animals. Without wishing to be bound by theory, the provision of a safe and effective vaccine for treating non-human animals will impact on human health, for example, the eradication of rabies from dog populations would be expected to eliminate human disease.

Importantly, as discussed herein, administration may be via baits for protecting and treating wildlife and stray animals that can be infected by lyssaviruses as well as domesticated pets. In a preferred aspect, administration is via baits for protecting and treating dogs.

In one aspect, the immune-stimulating composition of the invention is a live attenuated viral vaccine. A live attenuated viral vaccine requires the virus to be able to replicate in vitro and in vivo. Importantly therefore, from the perspective of being able to utilise a lyssavirus particle of the invention in a vaccine, the lyssavirus is able to replicate in vivo and in vitro, facilitating vaccine production. In one aspect when the lyssavirus or lyssavirus virion has decreased inhibition of IFN-dependent signalling, immune stimulation is facilitated.

In one aspect, the present invention provides a pharmaceutical composition comprising a lyssavirus or a lyssavirus virion as described herein, and a pharmaceutically acceptable carrier.

Pharmaceutically acceptable carriers or diluents contemplated by the invention include any diluents, carriers, excipients, and stabilizers that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides;

proteins, such as plasma albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The live attenuated viral vaccines described herein can be administered without the need for formulation with an adjuvant. Without wishing to be bound by theory, because the live attenuated viral vaccines described herein are able to infect and replicate within cells and preferably induce innate and adaptive immune responses (e.g. replication-competent vaccines can strongly activate cellular as well as humoral responses) to a high level, in a preferred form there is a reduced need for formulation with an adjuvant (e.g. the attenuated viral vaccine possesses adjuvanting potential), reduced need for multiple administration and/or a reduced need for administration with rabies immunoglobulin (e.g. in therapeutic vaccination of humans). Because of the potential to generate stronger and more rapid responses, live vaccines may also extend the window during which therapeutic vaccination can be successful in infected humans or animals.

Alternatively, the live attenuated viral vaccine of the invention is optionally formulated with an adjuvant. An adjuvant is a pharmacological or immunological agent that modifies the effect of other agents. In terms of their inclusion in vaccine formulations, adjuvants enhance the recipient's immune response to the antigenic component of the vaccine.

Adjuvants suitable for use in the vaccine of the invention include but are not limited to aluminium salts, hydroxide, paraffin oil, calcium phosphate hydroxide, beryllium, bacterial products, monophosphoryl lipid A, Freund's complete adjuvant, and Freund's incomplete adjuvant, and combinations thereof.

In one aspect, the present invention provides a use of a lyssavirus virion as described herein in the manufacture of a medicament for treating and/or preventing lyssavirus infection in a subject.

In one aspect, the present invention provides a method of treating and/or preventing lyssavirus infection in a subject, said method comprising administering to the subject a therapeutically effective amount of a pharmaceutical composition as described herein.

As used herein, a “therapeutically effective amount” of a pharmaceutical composition refers to an amount of a pharmaceutical composition which, when administered, produces an anti-lyssavirus immune response in a subject. In one aspect, the immune response can protect the subject from infection by the lyssavirus if they are exposed to it.

As used herein the terms “treating” and “treatment” include ameliorating a sign or symptom of lyssavirus infection, and refers to any observable beneficial effect following administration of a composition comprising a lyssavirus described herein. The beneficial effect can be evidenced, for example, by a delayed onset of clinical symptoms of the lyssavirus infection in a susceptible subject, a reduction in severity of some or all clinical symptoms of the lyssavirus infection, a slower progression of the lyssavirus infection, an improvement in the overall health or well-being of the subject, or by other parameters well known in the art that are specific to the lyssavirus infection.

As used herein the terms “protecting’ and “protection” include partial protection from infection with lyssavirus, or lyssavirus infection associated mortality, and also include protection requiring booster vaccinations or other suitable treatment, including post-exposure treatments.

In the case of subjects already exposed to or infected by a lyssavirus, the “therapeutically effective amount” refers to an amount that enhances the subject's immune response to clear the lyssavirus from the subject and to protect from future infection. The production of an anti-lyssavirus immune response in a subject can be confirmed using techniques known in the art; for example, by measuring antibodies, or by determining a subject's ability to survive subsequent challenge, or by measuring viral load after challenge.

A lyssavirus virion, or a pharmaceutical composition comprising a lyssavirus virion of the invention may be given to subjects at risk of infection from a lyssavirus, thereby functioning as a preventative (or prophylactic) vaccine, preferably for at least rabies virus and/or viruses of the same serotype (e.g. Australian bat lyssavirus). As a result of the vaccination the subject develops immunity to infection from the lyssavirus. The invention therefore provides a method of protecting a subject from infection with a lyssavirus comprising administering an effective amount of lyssavirus virion, or a pharmaceutical composition comprising a lyssavirus virion according to the invention described herein. Administration of the lyssavirus virion, or a pharmaceutical composition comprising a lyssavirus virion as described herein, induces an anti-lyssavirus immune response that subsequently protects the human or non-human subject from infection by the lyssavirus if they are exposed to it.

Alternatively, the lyssavirus virion, or a pharmaceutical composition comprising a lyssavirus virion of the invention may be given to subjects who have, or are suspected to have been, exposed to lyssavirus, thereby functioning as a therapeutic vaccine. The vaccine enhances the subject's own immune response capabilities while also protecting the subject from re-infection by a lyssavirus. Accordingly, in this aspect of the invention, there is provided a method of treating a subject exposed to, or suspected as having been exposed to, a lyssavirus comprising administering to the subject an effective amount of a lyssavirus virion, or a pharmaceutical composition comprising a lyssavirus virion as described herein.

The methods described herein optionally include screening the subject for suspected lyssavirus infection prior to administration of the live attenuated viral vaccine.

Subjects to which the lyssavirus virion, or a pharmaceutical composition comprising a lyssavirus virion as described herein may be administered are humans and any other warm blooded animals, particularly domesticated animals such as dogs and cats (including stray dogs and cats), and wildlife including but not limited to bats, skunks, foxes, otters, ferrets, horses, other farm animals, monkeys, wolves, wild dogs, coyotes, dingoes, raccoons and opposums.

The pharmaceutical composition may be conveniently presented in unit dosage form and prepared using conventional pharmaceutical techniques. Such techniques include the step of bringing into association the active ingredient and the pharmaceutical carrier(s) or excipient(s).

Formulations suitable for parenteral administration include aqueous and non-aqueous sterile injection solutions which may contain anti-oxidants, buffers, bacteriostats and solutes which render the formulation isotonic with the blood or a cell of a tissue of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) or equivalent stable condition requiring only the addition of a sterile liquid carrier, for example, water for injections, immediately prior to use. Extemporaneous injection solutions and suspensions may be prepared from sterile powders, granules and tablets commonly used by one of ordinary skill in the art.

In certain embodiments, unit dosage formulations are those containing a dose or unit, or an appropriate fraction thereof, of the administered ingredient. It should be understood that in addition to the ingredients particularly mentioned above, formulations encompassed herein may include other agents commonly used by one of ordinary skill in the art.

The compositions provided herein, including those for use as immunogenic compositions, may be administered through different routes, such as oral, including buccal and sublingual, rectal, parenteral, aerosol, nasal, intramuscular, subcutaneous, intradermal, transdermal (e.g. patch based delivery) and topical. They may be administered in different forms, including but not limited to solutions, emulsions and suspensions, microspheres, particles, microparticles, nanoparticles, and liposomes. In some embodiments, the immunogenic compositions are administered orally.

In one preferred aspect, the mode of administration is oral administration.

Administration may be via baits for protecting and treating wildlife and stray animals that can be infected by lyssaviruses as well as domesticated pets.

In a preferred aspect, administration is via baits for protecting and treating dogs.

Preferably, the lyssavirus is rabies virus.

The volume of administration will vary depending on the route of administration. Those of ordinary skill in the art can determine appropriate volumes for different routes of administration.

Administration can be accomplished by single or multiple doses. The dose administered to a subject in the context of the present disclosure should be sufficient to induce a beneficial therapeutic response over time, such as to prevent lyssavirus infection or the development of rabies. The dose required may vary depending on, for example, the age, weight and general health of the subject.

Preferably, administration is performed using a single dose. Benefits of single dose vaccines include cost effectiveness, and ease of delivery, in addition to immune stimulation.

The amount of pharmaceutical composition in each dose is selected as an amount that induces an immune-stimulating response without significant, adverse side effects. Such amount will vary depending upon which specific composition is employed and how it is administered. Initial doses may range from about 1 μg to about 1 mg, with some embodiments having a range of about 10 μg to about 800 μg, and still other embodiments a range of from about 25 μg to about 500 μg. Following an initial administration of the immunogenic composition, subjects may receive one or several booster administrations, adequately spaced. Booster administrations may range from about 1 μg to about 1 mg, with other embodiments having a range of about 10 μg to about 750 μg, and still others a range of about 50 μg to about 500 μg. Periodic boosters at intervals of 1-5 years, for instance three years, may be desirable to maintain the desired levels of protective immunity. In preferred embodiments, subjects receive a single dose of a pharmaceutical composition as described herein, and in a preferred embodiment, subjects receive a single dose of a pharmaceutical composition as described herein delivered orally.

Throughout the description and the claims of this specification the word “comprise” and variations of the word, such as “comprising” and “comprises” is not intended to exclude other additives, components, integers or steps.

### EXAMPLES

### Example 1: Materials and Methods

Construction of Expression Vectors and Mutagenesis

GB1-fused STAT1 expression vectors were created by inserting the genes corresponding to full-length STAT1 (4-750) and STAT1-CCD-DBD (136-490) into the pGEV2 vector which generates a GB1-fusion protein with a thrombin-cleavable linker between GB1 and the expressed protein, and a C-terminal His6-tag. Full-length STAT1 was also cloned into pGEX6P3 to produce GST-STAT1.

The NiP-CTD gene (residues 186-297) with the C-terminal cysteine (Cys297) mutated to serine was cloned into the Ndel-EcoRl sites of a pET28a vector as a His6-tagged protein with a TEV cleavage site. GFP-fused NiP-CTD (GFP—NiP-CTD) was similarly cloned and comprised an ultra-stable, monomeric GFP along with the NiP-CTD sequence. DNA encoding the N-peptide (aa 363-414) of the N protein was inserted into a pGEX-6P-3 vector using BamHl-Xhol restriction sites, with an N-terminal GST-tag followed by a PreScission protease cleavage site.

Mutations were introduced into the N-peptide or NiP-CTD constructs using PrimeSTAR Max DNA Polymerase (Takara) following the manufacturer's instructions. Mutagenesis primers were designed and the PCR mixture digested with Dpnl for 1.5 h at 37° C. before transforming into chemically competent Top10 E. coli cells. The plasmid bearing the mutation was isolated from a single colony and confirmed by sequencing.

For mammalian cell expression of P proteins, full length Ni- and CVS—P proteins, CVS PΔ30 and mutant Ni—P were cloned into pEGFP-C1 as described elsewhere.

Protein expression and purification The GB1-STAT1 constructs were expressed in E. coli BL21 (DE3) in 2YT autoinduction media at 16° C. with shaking at 225-230 rpm. GB1-STAT1 variants were purified as follows. Pellets from 500 mL cultures were resuspended in 50 mL extraction buffer (50 mM Na₂HPO₄, 300 mM NaCl, 10 mM imidazole, pH 7.4), homogenized and lysed (Avestin EmulsiFlex C3 cell crusher). Following centrifugation (13,000 g, 30 min, 4° C.), the supernatant was filtered (0.22 μm filter), applied to 5 mL Talon^{R} metal-affinity resin (Clontech, TAKARA) packed in a gravity-flow column. After 90 mins of binding at 4° C., unbound proteins were discarded by washing (100 mL of extraction buffer); GB1-fusion proteins were eluted (80 mL of 50 mM Na₂HPO₄, 300 mM NaCl, 300 mM imidazole, pH 7.4). Eluent fractions were concentrated to 1.5 mL using Amicon Ultra-15 with MWCO 10 kDa (truncates of GB1-STAT1) or 30 kDa (full-length GB1-STAT1) and further purified by size exclusion chromatography (SEC) on a HiLoad™ 16/60 Superdex™ 200 prep grade column pre-equilibrated with 50 mM Na₂HPO₄, 100 mM NaCl, 1 mM DTT, pH 6.8 and run at a flow rate of 1 mL/min. Eluted 1 mL fractions were collected, pooled and reconcentrated.

GST-tagged STAT1 was purified similarly to above except after centrifugation the filtered supernatant was bound to 5 mL of Glutathione Sepharose 4 Fast Flow resin (GE Healthcare) pre-equilibrated in 50 mM Na₂HPO₄, 300 mM NaCl, 1 mM DTT, pH 7.4. After 90 mins, unbound proteins were removed and GST-STAT1 was eluted (80 mL of 50 mM Na₂HPO₄, 300 mM NaCl, 10 mM reduced glutathione, 1 mM DTT, pH 7.4); cleaved overnight with 3C protease (100 μl of purified 96 μM GST-fused 3C protease) at 4° C.; concentrated to 1.5 mL and further purified with SEC as described above.

Unlabelled NiP-CTD, mutants of NiP-CTD and GFP—NiP-CTD were expressed similarly to the GB1-STAT1 constructs; whereas ¹⁵N-labelled NiP-CTD and mutants were expressed in an autoinduction media using ¹⁵NH₄Cl as a sole nitrogen source. To label NiP-CTD mutants with ¹³C and ¹⁵N isotopes cells were grown in N-5052 supplemented with 1 g/L of ¹⁵NH₄Cl (Sigma-Aldrich) and 3 g/L of D-[¹³C] glucose (Sigma-Aldrich) as sole sources of nitrogen and carbon. Cells were grown at 37° C. until an OD₆₀₀ of 0.6-0.7, transferred to 16° C. and induced with 0.4 mM IPTG and protein expressed overnight with shaking at 225-230 rpm. To express ²H, ¹⁵N-labelled NiP-CTD, cells were grown in N-5052 medium prepared with 1 g/L of ¹⁵NH₄Cl and 2 g/L of D-[²H]-glucose (Cambridge Isotope Laboratories) in ²H₂O (Sigma-Aldrich). A 5 ml pre-culture was prepared from a single colony incubated at 37° C. for 6-7 h. Cells were then centrifuged, washed and resuspended into N-5052 media prepared in ²H₂O and with ¹H₆-glucose as the carbon source to make an overnight culture. Cells adapted to the deuterated media were pelleted, washed and diluted in N-5052 media supplemented with D-[²H]-glucose. After adapting to 16° C. protein expression was conducted overnight after inducing at OD600 of 0.7˜0.8 by adding 0.4 mM IPTG. All NiP-CTD proteins were purified over Talon® metal-affinity resin, and after cleavage with TEV protease subjected to SEC. A HiLoad™ 16/60 Superdex™ 75 prep grade column was used for NiP-CTD; whereas a HiLoad™ 16/60 Superdex™ 200 prep grade column was used for GFP—NiP-CTD. Further details of the purification of NiP-CTD are described in.

Mutants of NiP-CTD were purified as described above for wild-type. To assess the effect of mutation on solubility of expressed protein, similar volumes of bacterial culture expressing each mutant were lysed and fractionated into soluble supernatant and insoluble debris, and separated by SDS-PAGE. Solubility index was calculated using the band intensity on the gel measured using Image Lab 5.2.1 (Biorad).

¹⁵N-labelling of N-peptide (N protein, residues 363-414, S389E) of RABV was conducted similarly to NiP-CTD, except expression was as a GST-fusion prepared with the pGEX6P3 vector. To purify ¹⁵N-labelled N-peptide, cells pellets were processed as described for NiP-CTD purification except the extraction buffer was 50 mM sodium phosphate, 300 mM NaCl, 1 mM DTT at pH 7.5. Following cell crushing and centrifugation the clear supernatant was bound to 5 ml of Glutathione Sepharose High Performance resin packed into a gravity-flow column (GE Healthcare) at 4° C. After 2 h, unbound material was removed by washing with 100 mL extraction buffer. On-column cleavage was conducted with 3C protease (100 μL of purified in-house GST-fused 3C protease with a concentration of 96 μM) over 4 h at 4° C. Cleaved peptide was eluted from the column by gravity flow by washing with 20 mL of extraction buffer. After concentrating to 5 mL using Amicon Ultra-15 (MWCO 3 kDa) the peptide was further purified using reverse-phase high performance liquid chromatography (RP-HPLC) with an Agilent Zorbax 300SB—C18 column using buffer A 0.1% trifluoro-acetic acid, Buffer B 100% acetonitrile with 0.1% trifluoro-acetic acid. Collected fractions were pooled, freeze dried and stored at −20° C. for future use. The final yield obtained was 3-5 mg/L of bacterial culture.

Circular Dichroism (CD) Spectrophotometry

CD spectrophotometry was performed using a 410 SF Circular Dichroism spectrometer (AVIV Biomedical, Lakewood, N.J.). Measurements used a quartz cuvette with a 0.1 cm path length and 0.1-0.2 mg/mL of protein in 50 mM Na₂HPO₄, 100 mM NaCl, 1 mM DTT, pH 6.8 at 25° C. A wavelength range of 190-260 nm was scanned with an increment of 0.5 nm and an averaging time of 1.0 sec. For each protein three scans were recorded, averaged and subtracted from three averaged buffer scans. Mean residue ellipticity (MRE) (deg.cm².dmol⁻¹) was calculated using MRE=θ.MRW/10.1.c, where θ is the ellipticity (millidegrees), I is the pathlength (cm), c is the protein concentration (mg/mL) and MRW is the Mean Residue Weight calculated as MRW=Mr/(N-1), where Mr is the MW of the protein (Da) and N is the number of residues. Secondary structure analysis of STAT1 variants and NiP-CTD (wild-type and mutants) were conducted in DichroWeb using the program CDSSTR.

Thermal unfolding of NiP-CTD (wild-type and mutants), was measured by raising the sample temperature from 20 to 90° C. at a rate of 1° C./min. Thermal unfolding transitions and mid-point melting temperature (Tₘ) were calculated by plotting normalized ellipticity values at 222 nm as a function of temperature and fitted to a two-state transition (equation 1), assuming no change to heat capacity for folded and unfolded, and correcting for pre- and post-transition changes:

$$Y = \frac{( {Y}_{N} + {β}_{N} T ) + ( {Y}_{D} + {β}_{D} T ) {e}^{- ( Δ {H}_{T m} ( 1 - ( \frac{T}{T m} ) ) / R T}}{1 + {e}^{- ( Δ {H}_{T m} ( 1 - ( \frac{T}{T m} ) ) / R T}} \qquad ( 1 )$$

where Y is the observed ellipticity at a given temperature, Y_{N} (Y_{D}) and β_{N} (β_{D}) are the slopes and intercepts of the pre- and post-transition slopes; T is temperature (° C.), Tₘ the mid-point melting temperature, and ΔH_{Tm} is the enthalpy at Tₘ.

Analytical Ultracentrifugation Characterization of NiP-CTD and STAT1 Interaction

Sedimentation Velocity Analytical Ultracentrifuge (SV-AUC) experiments were conducted on a Beckman Optima XL-I AUC equipped with an An50 Ti rotor (Beckman Coulter, Ind.). All protein samples were dialysed against SV-AUC buffer (50 mM Na₂HPO₄, 100 mM NaCl, 2 mM TCEP, pH 6.8) and the buffer was used as reference for each experiment. Samples containing GB1-STAT1 proteins at varying molarities were loaded into the sample compartments of Epon double-sector centrepieces, with buffer in the reference compartment. Samples were centrifuged at a rotor speed of 50,000 rpm, at 20° C., and monitored continuously at a wavelength of 280 nm. Fluorescence-detected SV-AUC (FDS-AUC) experiments were conducted in a Beckman Optima XL-A AUC equipped with a fluorescence-detection system (AVIV Biomedical, Lakewood, N.J.). The concentration of GFP—NiP-CTD was kept constant at 10 μM while the concentrated GB1-STAT1 variants were diluted in SV-AUC buffer ranging from 5 to 40 μM. Samples were centrifuged at a rotor speed of 50,000 rpm, at 20° C. and monitored continuously. Data were fitted in the program SEDFIT using 100 sedimentation coefficient increments ranging from 0 to 15 S, with a regularization parameter of p=0.95. The frictional ratios were fitted, and for the fluorescence-detected experiments, meniscus positions were also fitted.

NMR Data Acquisition

For NMR experiments, NiP-CTD and STAT1 samples were prepared were dialyzed in the same buffer (50 mM Na₂HPO₄, 100 mM NaCl, and 1 mM DTT, pH 6.8) prior to making final samples in 10% D₂O/90% H₂O. All NMR data were acquired at 25° C. on a Bruker Avance IIIHD 700 MHz spectrometer equipped with a triple resonance cryoprobe. The near-complete assignment of the ¹H, ¹³C, ¹⁵N resonances of wild type NiP CTD have been reported elsewhere (Biological Magnetic Resonance Bank, accession code 27498). To assign the backbone (HN, ¹⁵N, ¹³Cα, ¹³Cβ, ¹³C′) resonances of the NiP-CTD W265G/M287V and F209A/D235A mutants, data were collected for the 3D experiments (HNCO, HN(CA)CO, HNCACB, HNCOCACB) using uniformly ¹³C-¹⁵N labelled protein. 2D ¹⁵N, ¹H Heteronuclear Single Quantum Coherence (¹⁵N,¹H HSQC) spectra were collected using traditional approaches whereas, all 3D spectra were recorded using 10% non-uniform sampling (NUS) and Poisson gap sampler. Spectra were reconstructed with the compressed sensing algorithm using qMDD, processed using NMRPipe, and analysed with NMRFAM-SPARKY.

Interactions between ¹⁵N-labelled NiP-CTD and the GB1-STAT1 were monitored by acquiring spectra of NiP-CTD and GB1-STAT1 at a ratio of 1:1. For transferred cross-saturation experiments we prepared samples by mixing 500 μM uniformly labelled ²H-¹⁵N NiP-CTD with 50 μM GB1-STAT1 or GB1-STAT1-CCD-DBD in 50 mM sodium phosphate, 100 mM NaCl, 1 mM DTT at pH 6.8 in 90% ²H₂O/10% H₂O. Prior to mixing with STAT1, ²H-¹⁵N NiP-CTD was kept in the same buffer for 8 h at room temperature and then 2 days at 4° C. to allow amide exchange to reach equilibrium. The 2D ¹⁵N, ¹H TROSY-HSQC pulse scheme and WURST ¹H-saturation pulse (15 ms, 2800 Hz band-width) used in our study were as described. The data were acquired with 25% non-uniform sampling and Poisson gap sampler with interleaved rows for on- and off-saturation; spectral widths of 12 ppm in ¹H (2048 data points), and 27 ppm in ¹⁵N (512 data points). The WURST saturation of the aliphatic protons NiP-CTD was 2 s with a saturation frequency set at 0.9 ppm for on-resonance and −50 ppm for off-resonance. Each transferred cross-saturation experiment was acquired in 6 to 13 h with 64 to 112 scans per row and a recycle time between scans of 1 s. Spectra were reconstructed with the compressed sensing algorithm using qMDD and processed using NMRPipe.

To monitor the binding of RABV N-peptide to wild-type and mutant NiP-CTD, 2D ¹⁵N, ¹H HSQC-monitored titrations were conducted using 50 μM of ¹⁵N-labelled N-peptide with an increasing concentration (25, 50, 100, 200, 400, 500 μM) of unlabelled NiP-CTD variants. During the titration, the volume of the NMR sample was kept within a variation of 10%. The average chemical change was determined from:

Δδppm=((Δ¹HN)²+(0.15Δ^{15N}N)²))^{1/2} (2)

The dissociation constants (K_{D}) were measured using well-resolved peaks that showed the largest shifts and remained in fast exchange during the titration. Data were fitted to a non-linear curve assuming a two-state exchange (xcrvfit 4.0.12; Boyko and Sykes, University of Alberta, www.bionmr.ualberta.ca).

Hydrogen-deuterium exchange of wild-type and mutant NiP-CTD was monitored via the acquisition of 2D ¹H-¹⁵N HSQC spectra at 25° C. and pH 6.8 on a 600 MHz Bruker Avance III spectrometer. Exchange was initiated by passing a 250 μM sample over an illustra NAP-5 column pre-equilibrated in 50 mM Na₂HPO₄, 100 mM NaCl, 1 mM DTT, pH 6.8, 100% D₂O. Data were acquired with spectral widths of 13 ppm in ¹H (2048 data points) and 26 ppm in ¹⁵N (256 data points). NUS was used for acquisition with 25% sampling. For each spectrum 16 scans were acquired per ¹⁵N data point resulting in acquisition times of 20 minutes. Acquisition of the first spectrum occurred after 8 mins following initial exchange. Exchange rates (kₐ) were determined by fitting the to a single exponential,

I=e^{-k}α^{t} (3)

where I is the peak intensity, kₐ, the exchange rate and t is time. The difference in free energy of exchange (δΔG kJ/mol) between wild type and mutant protein was determined from

$$δ Δ G = - R T ln ( \frac{{k}_{a 1}}{{k}_{a 2}} ) \qquad ( 4 )$$

where R is the gas constant, T is temperature (K) and kₐ₁ and kₐ₂ are the exchange rates for the same proton.

Mammalian Cell Culture

HEK-293-T cells were cultured at 37° C., 5% CO₂, in Dulbecco's minimal essential medium (DMEM) supplemented with 10% fetal calf serum (FCS).

Luciferase Reporter Gene Assays

Dual luciferase assays to measure induction of type-I IFN or type-I IFN signalling are performed as previously described. Briefly, HEK-293-T cells cultured in a 24-well plate are co-transfected with pEGFP-C1 constructs encoding full-length wild-type or mutated P protein, pRL-TK (Promega) (which constitutively expresses Renilla luciferase) and either pISRE-Luc (Stratagen) or pGL3-IFNb, which express firefly luciferase under the control of an ISRE promoter or IFNβ promoter respectively, using Fugene (Promega) or Lipofectamine 2000 (ThermoFisher), according to the manufacturer's instructions.

For assays of IFN signalling, cells are treated 7 h post-transfection with 1000 U/ml IFNα (PBL Interferon Source) for 16 h before analysis by dual luciferase assay. Relative luciferase activity is determined as previously described, by normalising firefly luciferase activity to Renilla luciferase activity and calculating the normalised values relative to those for positive control samples (cells expressing PΔ30 and treated with IFN) (FIG. 5).

For IFN induction assays, cotransfection includes RIG-I-flag (to activate RIG-I signalling) and/or pUC-19 to equalise total DNA transfected for samples without transfection of RIG-I or P protein plasmid. Luciferase activity is measured 24 h post-transfection by dual luciferase assay. Relative luciferase activity is determined as above, by normalising firefly luciferase activity to Renilla luciferase activity and calculating the normalised values relative to those for positive control samples expressing pUC-19 and RIG-I-flag without P protein.

Minigenome assays are performed as described previously. Briefly, HEK-293-T cells seeded in 12-well plates are transfected with 0.4 μg pRVDI-luc, 0.6 μg pC-RN, 0.2 μg pC-RL, and 0.1 μg pEGFP-C1 encoding the wild-type and mutant P proteins. Cells are lysed 48 h later and analyzed for firefly luciferase activity as described above.

For analysis of IFN signalling in infected cells, cells expressing luciferase under the control of an ISRE (for example STING-37 reporter cells) are infected with wild-type or mutated recombinant virus (for example Tha, CE-NiP virus), or mock infected. Following incubation, cells are treated without or with IFNα and analysed using the Firefly Luciferase kit (Promega).

Confocal laser scanning microscopy (CLSM) analysis of STAT1 localization Cos-7 cells are seeded onto coverslips and transfected the next day with pEGFP-C1 plasmids expressing wild-type or mutant Ni—P using Lipofectamine (ThermoFisher) according to the manufacturer's instructions. 16 h post-transfection. cells are treated without or with IFNα for 30 min prior to fixation with 3.7% formaldehyde for 10 min and permeabilization with 90% methanol for 5 min. Cells are immunostained with anti-STAT1 antibody followed by Alexa Fluor-568 conjugated secondary antibody. Coverslips are mounted onto glass slides using Mowiol mounting solution. Cells are imaged by CLSM.

Immunoprecipitation (IP) and Immunoblotting Assays

Co-immunoprecipitation (co-IP) assays are performed as previously described. Briefly, COS7 cells are transfected to express GFP-fused P proteins before incubation overnight in DMEM with 0.5% FCS. Cells are then incubated in serum-free DMEM (1 h) before treatment with 1000 U/ml IFNα. Following incubation, cells are washed twice with PBS and harvested into cell lysis buffer (10 mM Tris-HCl, 150 mM NaCl, 0.5 mM EDTA, 0.5% NP-40, pH 7.5). Lysate is passed through a 27G needle 10 times, and incubated on ice (30 min) before clearing by centrifugation (12000 g, 10 min, 4° C.).

10% of the lysate (‘Input’ sample) is solubilised in SDS-PAGE loading buffer and the remainder subjected to co-IP using the GFP-Trap system (Chromotek) according to manufacturer's instructions, before elution using SDS-PAGE loading buffer. Input and co-IP samples are separated by SDS-PAGE before western blotting and analysis using anti-pY-STAT1 antibody, anti-STAT1, and anti-GFP, followed by HRP-conjugated secondary antibodies and detection using Western Lightening ECL reagents (Perkin Elmer) and a Gel Doc™ XR+Gel Documentation System.

Reverse genetics and viral production Recombinant viruses are generated as previously described using plasmid carrying the genome of rabies virus. Mutations were introduced to the CE-NiP-WT genome plasmid by overlap PCR as previously described in Wiltzer et al., (JID, 209: 11(1) 1744), and recombinant virus rescued in BHK/T7-9 cells. Viral stocks were prepared in NA cells and titers were determined by focus formation assay to calculate focus forming units (ffu)/mL again as previously described in Wiltzer et al.

In Vivo Experiments

Pathogenesis experiments are performed as previously described; briefly, BALB/C mice are infected by intramuscular injection of 1000 FFUs of recombinant RABV, and monitored for 21 days. Mice are sacrificed when late infection symptoms appear (humane endpoint).

Statistical Analysis

Prism version 6 software (Graphpad) is used for statistical analysis to calculate p-values using Student's t-test (unpaired, two-tailed). To calculate p-values for survival curves the log-rank (Mantel-Cox) test is used.

### Example 2: Expression, Purification and Characterization of STAT1

A number of structural studies of STAT1 have used truncated versions of the protein, suggesting problematic expression, purification and solubility. To assess P-protein-STAT1 interaction, full-length STAT1 and a truncate consisting of the coiled-coil and DNA-binding domains (STAT1-CCD-DBD) were expressed as GST fusions. After purification by affinity chromatography the yield of both proteins was 5 mg from 1 L of culture. Following removal of the GST-tag and purification through SEC the yield was less than 2 mg. SDS-PAGE showed aggregates of more than 50% of STAT1 proteins eluting in the void volume of SEC. Furthermore, both purified STAT1 and STAT1-CCD-DBD almost immediately commenced precipitation at room temperature or within 8 hours at 4° C., indicating poor stability without the tag.

To improve yield and solubility, the GB1 fusion tag as a solubility-enhancer tag was tested. Full-length STAT1, expressed with an N-terminal GB1-tag and a C-terminal His₆-tag (GB1-STAT1), showed >80% of the expressed fusion protein was present in the soluble fractions after cell lysis. Similar results were observed for the fusion GB1-STAT1-CCD-DBD. The total amount of both proteins obtained after affinity chromatography and SEC was about 30 mg/L of culture. In contrast to the cleaved GST-STAT1 fusion, SEC showed little aggregation (FIG. 6), therefore the GB1-tag markedly enhances the expression, stability and solubility of STAT1, enabling >15-fold improvement in yield compared to GST-tag purification and subsequent removal of the GST.

To determine the potential structural impact of GB1-tagging and truncation of STAT1, CD spectrophotometry was used to estimate secondary structure (FIG. 7). Both constructs showed spectra consistent with folded proteins. The experimental secondary structure values for GB1-STAT1 and GB1-STAT1-CCD-DBD fit well with those calculated (FIG. 7c) suggesting correct folding. SV-AUC experiments were conducted to characterize the hydrodynamic properties of the proteins and their ability to form expected protein-protein interactions. Sedimentation coefficient distributions c(s) indicated that GB1-STAT1 (FIGS. 8a and 9) forms a major species and two minor species at three concentrations (30, 50, 80 μM), where the major peak had a weight average coefficient of ˜6.5 S, corresponding to the expected GB1-STAT1 dimer, and an estimated molecular weight of 186.8 kDa (consistent with the theoretical mass of 191 kDa for the dimer). The smaller peaks (˜3.7 S, ˜9.5 S) likely correspond to small amounts of monomeric and multimeric STAT1. The frictional ratio for GB1-STAT1 was 1.7, suggesting an elongated shape. Sedimentation coefficients for GB1-STAT1-CCD-DBD (at 10 and 20 μM) (FIGS. 8b and 10) identified a major species with a weight average coefficient of ˜2.9 S corresponding to an estimated molecular weight of around 48.9 kDa, near the theoretical mass of monomeric GB1-STAT1-CCD-DBD (50 kDa), consistent with the absence of the N-terminal domain (ND) which stabilizes U-STAT1 dimerization. The frictional ratio for GB1-STAT1-CCD-DBD is 1.5, also indicating an asymmetrical shape.

### Example 3: Characterisation of the STAT1 Interacting Surface of the P-Protein

To characterise the signal transducer and activator of transcription 1 (STAT1) interacting surface of the P-protein, the present inventors examined complexes between the P-protein CTD and STAT1.

In brief, the inventors expressed and purified the P-CTD of the Nishigahara strain of RABV (NiP-CTD) tagged with GFP. FDS-AUC experiments were conducted on 10 μM GFP—NiP-CTD titrated with 5, 10 and 20 μM non-fluorescent GB1-STAT1 proteins (FIGS. 8c and 11), revealing a new peak at ˜7.4 S, attributed to a GFP—NiP-CTD/GB1-STAT1 complex. Titration using GB1-STAT1-CCD-DBD (FIGS. 8d and 12) revealed a sharp decrease of the GFP—NiP-CTD peak and appearance of a new species around 3.7 S. Thus, the STAT1 proteins interact with NiP-CTD. Assuming 1:1 binding and taking into account peak volumes of free and complexed GFP—NiP-CTD, the present inventors estimate an affinity for GB1-STAT1-CCD-DBD of approximately 10-20 μM (K_{D}).

The above data indicate that the STAT1 interacting surface of the Ni—P-protein is within the P-CTD, and involves the STAT1-CCD (Coiled-Coil Domain)-DBD (DNA Binding Domain. As the AUC experiments showed NiP-CTD binds weakly to U-STAT1, transferred cross-saturation NMR experiments using ¹⁵N, ²H-labelled NiP-CTD and ¹⁴N, ¹H-labelled GB1-STAT1-CCD-DBD or GB1-STAT1 (FIG. 13) were acquired. With STAT1-CCD-DBD, amide resonances from two regions of NiP-CTD (GLu200-Ser210, denoted region A, and Leu234-Lys239, region B, of SEQ ID NO: 1), which are separated in the protein sequence but proximal in the CTD structure, were significantly attenuated (FIG. 1). This data indicates that regions A and regions B form part of the STAT1 interacting surface of the P-protein, forming direct contacts with STAT1-CCD-DBD, while the distant region C forms distinct interaction involving the N and/or C terminal domains.

Experiments using GB1-STAT1 confirmed that these regions are attenuated, and additionally indicated attenuation of resonances of a third region, GIn275-VaL278 (region C) of SEQ ID NO: 1, indicative of an extensive interface between the NiP-CTD and STAT1 that could only be revealed using full-length STAT1 (FIG. 1A). This data indicates that region C forms part of the STAT1 interacting surface of the P-protein.

Previous experiments indicated that mutation of W-hole residues Trp265 and Met287 inhibit binding and antagonism of STAT1, and viral pathogenesis in vivo, suggesting that the W-hole might form part of the STAT1 interacting surface of the P-protein. Notably, our experiments indicate that binding does not involve the W-hole, which appears distant from the most attenuated residues (FIG. 1). Importantly, the indole NH of Trp265 is not attenuated at all (FIG. 13) supporting that the side-chain of this residue is not a part of the binding site. These data indicate that the W hole does not form part of the STAT1 interacting surface of the P-protein.

### Example 4: Amino Acid Substitutions in a STAT1 Interacting Surface of the P-Protein Alter STAT1 Antagonism

To validate the new proposed STAT1 binding sites of NiP-CTD, alanine point mutations were introduced into full-length Nishigahara P protein (Ni—P) and antagonism of STAT1 in mammalian cells assessed using an IFN-dependent luciferase reporter gene assay. Residues (FIG. 5) of the NiP-CTD were selected based on the degree of attenuation of resonances and surface exposure (FIG. 1). Assays included wild-type Ni—P (NiP-wt or wt) and CVS P protein (CVS-P or CVS-wt), which are functional for STAT1 antagonism, and CVS-P deleted for the C-terminal 30 residues (CVSD30 or Δ30). CVSD30 is a standard control, where the last two helices have been removed, resulting in a protein that lacks folded structure in the CTD and so is deficient in STAT1 targeting, replication, and most likely other CTD-associated functions.

Ni—P containing W-hole mutations W265G and M287V, as single mutations and combined were included, where the combined mutation strongly impairs STAT1 antagonism by CVS-P. As expected, wild-type Ni—P suppressed IFN-dependent signalling (indicated by reduced induction of luciferase), while CVSD30 was strongly defective (FIG. 5A). Ni—P W265G/M287V was also clearly defective, but this appeared to be to a lesser extent than the defect for CVSΔ30. Consistent with previous data for CVS-P mutated at corresponding residues, Ni—P W265G and M287V appeared to be partly defective.

Of the new mutants comprising amino acid substitutions, STAT1-antagonist function of A206E (FIG. 5B), F209A (FIG. 5A), D235K (FIG. 5B) and D235A (FIGS. 5A and B) was reduced, and in many cases to an extent similar or greater than that of W265G or M287V and exceeding that of other mutants tested (FIG. 5). To further test the importance of these residues, the double mutants F209A/D235A and A206E/D235K (FIGS. 5A and B) were prepared, thus combining one residue from each of region A and B (FIG. 1a); a mutant combining D235A/D236A (FIG. 5A), both of which are in region B, was generated to test the combined effect of mutation of the two neighbouring negatively charged side chains. While the data suggested further partial loss of STAT1-antagonist function by combination of D235A/D236A mutations, F209A/D235A and A206E/D235K resulted in ablation of IFN-antagonist function, being comparable to P130 (FIGS. 5A and B). Notably, despite a clear effect of W265G/M287V, the F209A/D235A and A206E/D235K, mutations were observed to cause a greater defect, suggesting that the latter mutations have a more potent impact on STAT1 interaction by NiP-CTD (FIG. 5A).

These data indicate that amino acid substitutions in a STAT1 interacting surface of the P-protein alter STAT1 antagonism. In particular, this data demonstrates one or more amino acid substitutions within or adjacent to a helix 1, a helix 2 and/or a helix 5 of the C-terminal domain of the P-protein can alter STAT1 antagonism.

This also demonstrates that one or more amino acid substitutions can modulate IFN antagonistic activity of the P-protein.

To confirm that the effect of F209A/D235A mutation on antagonism of IFN/STAT1-dependent signalling was due to an altered interaction with STAT1, the interaction was assessed in vitro by NMR. A 2D ¹H, ¹⁵N HSQC-monitored titration of ¹⁵N-labelled wild-type NiP-CTD with equimolar GB1-STAT1 was examined and resulted in an average of 52% loss of resonance intensity for 88 well-resolved peaks (FIG. 14a), as expected for binding of wild-type NiP-CTD to a dimer of GB1-STAT1 (forming a complex of ˜200 kDa). Intriguingly, despite clear (albeit incomplete) inhibitory effects on antagonist function of P protein in the IFN signalling assays (FIG. 5A), NiP-CTD W265G/M287V produced similar broadening patterns to wild-type, with an average of 41% loss of intensity for 83 well-resolved peaks (FIG. 14b). In contrast, titration of NiP-CTD F209A/D235A with GB1-STAT1, showed little broadening with an average of 7% loss of intensity for 85 well-resolved peaks (FIG. 14C). Thus, W265G/M287V mutations reduce, but do not ablate, binding to STAT1, consistent with incomplete effects on antagonism of STAT1 signalling, while F209A/D235A is strongly impaired, consistent with complete loss of IFN antagonist function. These data indicate that amino acid substitutions in the STAT1 interacting surface of the P-protein are able to interfere with the interaction of the P-protein with STAT1.

The original identification of P protein/STAT1 interaction showed that the STAT1-CCD-DBD is sufficient, indicating that STAT1 tyrosine phosphorylation (which occurs at a conserved residue in the C-terminal trans-activation domain, TAD; FIG. 7) is not required. However, the transferred cross-saturation data (FIG. 1) indicate a potential contribution by either the STAT1 C-terminal or N-terminal domain, indicating that virus-STAT complexes can involve a complex interface. Importantly, the data of Example 4 indicates that the residues involved in the P protein/STAT1 interaction are involved in STAT1 antagonism.

### Example 5: Amino Acid Substitutions in the W Hole Significantly and Globally Destabilize P-Protein

To assess the conformational integrity of the P-CTD containing F209A/D235A or W265G/M287V mutations, NiP-CTD containing single or double mutants were expressed and purified (FIG. 6). Importantly, while all single mutants and NiP-CTD F209A/D235A expressed as soluble proteins (>60%), NiP-CTD W265G/M287V expressed largely into the pellet fraction (10% in the cell-lysis supernatant), suggesting that the defective pY-STAT1 antagonism may be due to loss of proper folding.

To assess structural integrity of the W265G/M287V and F209A/D235A-mutated proteins, ¹³C, ¹⁵N labelled NiP-CTD mutants were generated, and assigned the ¹⁵N, NH, ¹³Cα, ¹³Cβ and C′ resonances by triple resonance experiments. As ¹³Cα and ¹³Cβ are sensitive to the secondary structure in proteins their differences using SSP were assessed, where positive and negative trends indicate α-helices and β-sheet, respectively. The secondary structure determined for wild-type NiP-CTD was consistent with the X-ray crystal structure of the CVS P-CTD (pdb ident: 1vyi), and there were no significant differences between the wild-type and mutant proteins (FIG. 4a). However, comparison of the ¹H-¹⁵N average chemical shift changes of W265G/M287V (FIG. 4b) and F209A/D235A (FIG. 4c) to wild-type NiP-CTD showed, as expected, changes near the sites of mutation, but also for W265G/M287V to regions distant from these sites in the folded structure suggesting an impact on the overall fold.

To assess protein stability, hydrogen-deuterium exchange experiments on wild-type, F209A/D235A and W265G/M287V NiP-CTD were conducted. Wild-type NiP-CTD showed a range of exchange rates, where amides of VaL238 and Leu268 were the most slowly exchanging, showing little exchange over 24 hours, whereas the amides of the C-terminal helix fully exchange within ˜30 mins. A number of other amides showed clear exponential decay permitting rates to be calculated (Table 1). In general, NiP-CTD F209A/D235A shows faster exchange for all amides compared to wild-type. For a number of well-resolved resonances where exchange rates for both the mutant and wild-type are readily determined, the differences in free-energy for unfolding (δΔG) between wild-type and F209A/D235A NiP-CTD were estimated to be 4.5 to 11.3 kj.moL⁻¹ (Table 1).

Remarkably, for NiP-CTD W265G/M287V the spectra showed complete hydrogen-deuterium exchange by the first spectrum (˜20 mins), suggesting destabilization of the hydrogen bond network of this mutant. In general, NiP-CTD A206E/D235K shows slower exchange for all amides compared to wild-type, suggesting that the A206E/D235K mutant is a slightly more stable structure under these experimental conditions (pH 6.8 and 25° C.).

TABLE 1
Hydrogen-deuterium exchange for WT, F209A/D235A and
A206E/D235K NiP-CTD^{a}

| | Secondary | Wild | F209A/ | A206E/ | δΔG |
|---|---|---|---|---|---|
| Residue | Structure | Type | D235A | D235K | (kJ · mol⁻¹) |
| I205 | α1 | 0.61 ± 0.07 | 58.8 ± .2.1 | 0.37 ± 0.02 | 11.3 |
| A206 | | 1.10 ± 0.06 | 79.3 ± 4.3 | 0.32 ± 0.02 | 10.6 |
| Y225 | β2 | 2.04 ± 0.03 | 53.0 ± 3.5 | 0.40 ± 0.02 | 8.1 |
| L230 | 3¹⁰ | 1.31 ± 0.08 | 44.7 ± 4.0 | 0.52 ± 0.02 | 8.7 |
| I237 | α2 | 2.30 ± 0.02 | 24.3 ± 0.1 | 1.19 ± 0.05 | 5.8 |
| K239 | | 2.30 ± 0.08 | 54.1 ± 2.5 | 1.26 ± 0.03 | 7.8 |
| V244 | Loop | 2.20 ± 0.13 | 60.9 ± 5.8 | 0.92 ± 0.02 | 8.2 |
| L263 | α4 | 0.53 ± 0.06 | 25.3 ± 1.2 | 0.23 ± 0.01 | 9.6 |
| G264 | | 2.4 ± 0.15 | 73.0 ± 4.8 | 1.08 ± 0.03 | 8.5 |
| W265 | | 1.2 ± 0.11 | 41.5 ± 3.2 | 0.45 ± 0.02 | 8.8 |
| L268 | | 0.47 ± 0.06 | 24.4 ± 0.9 | 0.14 ± 0.03 | 9.8 |
| L277 | α5 | 4.65 ± 0.34 | 28.7 ± 1.5 | 1.86 ± 0.05 | 4.5 |
| V278 | | 1.06 ± 0.08 | 17.1 ± 0.5 | 0.35 ± 0.15 | 6.9 |

^{a}Exchange rates of peptide HN groups with ²H₂O at pH 6.8 and 25° C. Rates and free energy differences of exchange (δΔG) for wild-type compared to F209A/D235A are given for protons that showed exchange where rates could be determined in both proteins. Similar experiments were performed on W265G/M287V NiP-CTD but fully exchanged prior to data acquisition (<20 min); data not shown as a result of destabilisation to the extent that values could not be determined.

Temperature unfolding, monitored by CD spectrophotometry was also performed (FIGS. 15 and 17). The full CD spectra at 25° C. of NiP-CTD show similar spectra for wild-type and F209A/D235A with estimates of similar content of secondary structure (FIG. 15). While estimated secondary structure content and the shape of the CD spectra of W265G/M287V were similar to wild-type and F209A/D235A, the depths of the CD signals were distinctly different. Unfolding of NiP-CTD, monitored at 222 nm, shows the wild-type and F209A/D235A mutant (FIG. 15), and A206E/D235K mutant (FIG. 17), fit to a two-site unfolding model with Tₘ of 57.0, 51.0 and 53° C. respectively.

However, the W265G/M287V mutant fits poorly to a two-state model and has an estimated Tₘ of 46° C. (FIG. 15). Collectively, the CD spectra, temperature unfolding and hydrogen-deuterium exchange data support that the conformation of NiP-CTD W265G/M287V is significantly and globally destabilized compared to the wild-type and the F209A/D235A and A206E/D235K mutants.

These data demonstrated amino acid substitutions in the W hole significantly and globally destabilize P-protein. These data also show that amino acid substitutions in the STAT1 interacting surface of the P-protein do not result in significant global destabilization of P-protein.

### Example 6: Amino Acid Substitutions in the STAT1 Interacting Surface of the P-Protein do not Abolish N-Protein Binding for Replication

Mutagenic studies suggest that the N-RNA binding site of the P protein is a cluster of positively charged residues (Lys211, Lys212 and Arg260) formed by the fold of the P-CTD. As this site is distal to the W-hole, mutations within the W-hole, as expected, did not substantially impact on replication. However, the N-RNA binding site is proximal to the newly identified binding region A (FIG. 1), and so could be affected by the NiP-CTD F209A/D235A mutations. To assess this, a peptide corresponding to a disordered region of N protein encompassing residues 363 to 414 (N-peptide), proposed to mediate P-CTD/N-RNA interaction in a SAXS model, was expressed. As phosphorylation of Ser389 in the N protein is reported to enhance this interaction, a phospho-mimetic mutation (S389E) was included in the peptide. 2D ¹⁵N, ¹H HSQC-monitored titrations of ¹⁵N-labelled N-peptide with NiP-CTD wild-type, F209A/D235A and W265G/M287V indicated significant chemical shift differences for residues between Thr375 to Gly397 of the N-peptide (FIG. 3) which fitted to a single-site binding curve (FIG. 3) showing a K_{D} of 88±4 μM for wild-type NiP-CTD and 122±11 μM for NiP-CTD F209A/D235A consistent with minimally perturbed structure. Titration of NiP-CTD W265G/M287V, however, gave a K_{D} of 249±29 μM (FIG. 3), more than a 2-fold loss of affinity, indicating that, while this mutant clearly will retain significant binding to N protein, permitting virus replication as previously observed, the data support the idea that W265G/M287V mutation affects the global structure of the P-CTD.

This data demonstrates that amino acid substitutions in the STAT1 interacting surface of the P-protein do not abolish N-protein binding, which is required for replication, demonstrating STAT1 binding and virus replication are spatially distinct.

To confirm that the observed binding to the N-peptide correlates with retention of replication function of the mutated proteins, a minigenome assay is used in which functional L-protein/P-protein/N-RNA interaction is indicated by luciferase activity. The effect of mutations including W265G/M287V mutation on replication function compared to wild-type is examined. Binding to N protein is sufficient to mediate efficient replication, as previously indicated by analysis of recombinant virus (e.g. by analysis of recombinant virus carrying mutated NiP, and minigenome assay of RABV CVS strain P protein).

Importantly, this data demonstrates that amino acid substitutions in the STAT1 interacting surface of the P-protein do not abolish RABV N protein interaction. Given the role of N protein, these data indicate that amino acid substitutions in the STAT1 interacting surface of the P-protein will not abolish polymerase cofactor function. This data also indicates that amino acid substitutions in the STAT1 interacting surface of the P-protein will not abolish replication function.

### Example 7: The Role of Amino Acid Substitutions in the STAT1 Interacting Surface of the P-Protein in RABV Attenuation

The present inventors have demonstrated previously that it is possible to introduce W hole mutations into RABV to inhibit STAT interaction (Wiltzer et al. JID, 209: 11(1) 1744), generating viable virus with growth kinetics indistinguishable from the parental strain in vitro, but which lacked IFN/STAT antagonist activity. This virus was highly sensitive to IFN and severely attenuated in vivo causing no lethality in mice, in contrast to the invariably lethal parental strain (data not shown).

Accordingly, to assess the role of the STAT1 interacting surface in infection, a recombinant RABV strain based on the CE-NiP strain (hereon referred to as CE-NiP-WT for wild type or CE-NiP-STAT (−) for mutant strains) and/or other relevant strains are used. In brief, mutations are introduced to the CE-NiP-WT genome plasmid by overlap PCR, and recombinant virus rescued in BHK/T7-9 cells. Viral stocks are prepared in NA cells, which are commonly used to prepare IFN-sensitive strains and titers aredetermined by focus formation assay to calculate focus forming units (ffu)/mL.

12 6-week-old female ddY mice (Japan SLC Inc.) per group are inoculated intracerebrally (i.c.) with 0.03 mL of diluent (mock) or diluent containing 104 ffu of virus. Mice are inspected for symptoms over 21 days. To measure viral titer in brains, mice are euthanized at 5 dpi and brains homogenized for analysis by focus formation assays.

For example, 12 ddY mice are i.c. inoculated with 10⁴ ffu of CE-NiP-WT or CE-NiP-STAT (−) (e.g. F209A/D235A mutant), and symptoms monitored over 21 days, including weight loss and severe neurological symptoms and mortality/a non-responsive end-point.

### Example 8: The Role of Amino Acid Substitutions in the STAT1 Interacting Surface of the P-Protein in STAT1 Interaction and STAT1 Nuclear Translocation

To confirm that effects on isolated protein interactions correlate with interactions in cells, co-IP and confocal laser scanning microscopy (CLSM) analysis were used. The original identification of P-protein/STAT1 interaction indicated that STAT1-CCD-DBD is sufficient to mediate binding in the absence of activation by tyrosine phosphorylation, as the tyrosine is in the transactivation domain (TAD). However, several studies show that efficient interaction detected by co-IP from cells requires IFN activation. Consistent with this, WT P-protein, but not PΔ30, co-precipitated STAT1 from cells treated with IFN for 0.5 h (FIG. 18A). In agreement with the lack of antagonistic function and binding in NMR, Ni—P F209A/D235A replicated the phenotype of P430, showing no detectable interaction (FIG. 18A, IP: lane FD).

CLSM analysis of the localization of immunostained STAT1 in cells expressing GFP-fused WT and mutant P-proteins indicated that, as expected, STAT1 rapidly accumulated into nuclei following IFN activation (0.5 h), and this was prevented by WT Ni—P (data not shown). Consistent with complete loss of STAT1-binding/antagonism, F209A/D235A Ni—P did not inhibit STAT1 nuclear translocation.

These data indicate that W265G/M287V-mutated protein retains significant capacity to bind STAT1 in vitro, but is strongly defective in antagonism of IFN/STAT1 signalling in cells (FIG. 5; FIG. 18A). Consistent with retention of binding, at least at early time points (0.5 h) of IFN treatment, W265G/M287V Ni—P suppressed IFN-dependent STAT1 nuclear translocation (data not shown). Co-IP assays also indicated W265G/M287V Ni—P interacted with STAT1 at 0.5-1 h IFN treatment when cellular pY-STAT1 levels are maximal; however, in multiple assays, binding was clearly reduced compared with WT P-protein. Furthermore, while WT P-protein retained STAT1 over extended periods (>16 h, as expected), binding was lost for W265G/M287V. P-protein causes accumulation of pY-STAT1 in cells, most likely due to retention in antagonistic complexes that prevent dephosphorylation by nuclear phosphatases, which ordinarily occurs from 0.5-1 h IFN-treatment as a negative regulatory mechanism (FIG. 18A).

Thus P-protein prevents normal phosphorylation/dephosphorylation recycling, which presumably enables sustained antagonism. These data indicate that W265G/M287V Ni—P is defective both for initial binding affinity and, consequently, retention of pY-STAT1, accounting for defective antagonism. Nevertheless, it clearly has residual STAT1 interaction compared with PΔ30 and F209A/D235A-mutated protein, explaining incomplete loss of antagonist function (FIG. 18). As the NMR data indicate that W265 and M287 do not directly contact residues of the novel binding regions, the effects of the mutations are likely indirect via conformational effects. In contrast, F209A/D235A mutation is as potent as P430, consistent with specific removal of critical STAT1 contacts.

### Example 9: P-CTD Interaction Directly Prevents STAT1-DNA Binding

The characterisation of mutations/substitutions specifically impacting regions A and B prevent P-protein from antagonising STAT1 transcription suggested that specific contacts with the CCD-DBD, and consequent inhibition of STAT1/DNA interaction, are critical.

To confirm the effects of WT and mutated P-CTD on STAT1/DNA binding in the absence of other cellular factors, we assessed pY-STAT binding to a DNA fragment containing GAS sequences. pY-STAT1 induced a strong concentration-dependent shift in electrophoretic mobility, with most of the DNA becoming arrested in the well (FIG. 18B); WT or mutated P-CTD alone caused no apparent shift of the DNA fragment mobility. Pre-incubation of pY-STAT1 with increasing amounts of WT P-CTD was clearly inhibitory, whereas F209A/D235A P-CTD had little to no impact at any concentration tested, supporting importance of regions A/B in binding to the DBD. W265G/M287V also lacked inhibitory activity, consistent with conformational effects reducing STAT1-binding capacity to a level insufficient to prevent DNA interaction.

These data demonstrate that the minimal STAT1-binding region of P protein, P-CTD, is sufficient to reduce DNA interaction. This supports a mechanism where formation of discrete P-CTD/STAT1 complexes, not requiring other viral/cellular proteins, antagonises STAT1/DNA interaction, consistent with the direct blockade of the DBD.

### Example 10: Amino Acid Substitutions in the STAT1 Interacting Surface of the P-Protein do not Interfere with Other Essential P-Protein Functions

Mutagenic studies suggest that the N-RNA binding site of P-protein is a cluster of basic residues (Lys211, Lys212, Arg260) formed by the P-CTD fold. Consistent with distal localization to the W-hole, W-hole mutations did not substantially impact on replication. However, the N-RNA binding site is proximal to region A, and so could be affected by F209A mutation. To assess this, a peptide (N-peptide) corresponding to a disordered region of N-protein (residues 363-414) was expressed, which was suggested to mediate P-CTD/N-RNA interaction in a SAXS model. 2D ¹⁵N, ¹H HSQC-monitored titrations of ¹⁵N-labelled N-peptide with WT and mutant P-CTDs showed significant chemical shift differences in N-peptide (FIG. 3 which fitted to a single-site binding curve (FIG. 3). WT and F209A/D235A, showed similar affinities, while W265G/M287V showed >2-fold loss of affinity (FIG. 3B). Thus, while W265G/M287V P-CTD clearly retains significant binding to N protein, permitting normal virus replication, these data support global structural effects of this mutant. To confirm that N-peptide binding correlates with replication function, a minigenome assay in which functional L-protein/P-protein/N-RNA interaction is indicated by luciferase activity was used (FIG. 3C). Despite ablation of STAT1 antagonist function, replication function of F209A/D235A P-protein was equivalent to that of WT.

Other than antagonizing IFN signalling, P-protein inhibits IFN induction in infected cells by antagonising the RIG-I-like receptor pathway. The responsible site(s) in P-protein are not known, but the C-terminal region 152-297 is suggested to be important. To determine whether F209A/D235A mutation impacts this function, we assessed RIG-I signalling using a reporter assay, finding no effect of F209A/D235A or W265G/M287V (the latter consistent with data for CVS-P (FIG. 3D). Thus, the new mutations specifically impact the STAT1 targeting arm of P-protein IFN antagonism.

1. An isolated lyssavirus phosphoprotein (P-protein) comprising one or more amino acid substitutions in a signal transducer and activator of transcription 1 (STAT1) interacting surface of the P-protein.

2. An isolated lyssavirus P-protein according to claim 1, wherein the interacting surface is within the C-terminal domain (CTD) of the P-protein.

3. An isolated lyssavirus P-protein according to claim 2, wherein the interacting surface is within the region corresponding to residues 186 to 297 of SEQ ID NO: 1.

4. An isolated lyssavirus P-protein according to any one of claims 1 to 3, wherein the one or more amino acid substitutions is within or adjacent to α helix 1, α helix 2 and/or α helix 5 of the of the C-terminal domain of the P-protein.

5. An isolated lyssavirus P-protein according to any one of claims 1 to 4, wherein the one or more amino acid substitutions interferes with the interaction of the P-protein with STAT1.

6. An isolated lyssavirus P-protein according to any one of claims 1 to 5, wherein the one or more amino acid substitutions modulates IFN antagonistic activity of the P-protein.

7. An isolated lyssavirus P-protein according to any one of claims 1 to 6, wherein the P-protein does not comprise an amino acid substitution in the W-hole of the P-protein.

8. An isolated lyssavirus P-protein according to any one of claims 1 to 7, wherein the one or more amino acid substitutions do not abolish polymerase cofactor function.

9. An isolated lyssavirus P-protein according to claim 8, wherein the one or more amino substitutions do not abolish N-protein binding.

10. An isolated lyssavirus P-protein according to claim 8, wherein the one or more amino substitutions are not in the N-protein interacting surface.

11. An isolated lyssavirus P-protein according to any one of claims 1 to 10, wherein the one or more amino acid substitutions are in the region corresponding to amino acid residues 203 to 277 of SEQ ID NO: 1.

12. An isolated lyssavirus P-protein according to any one of claims 1 to 11, wherein the one or more amino acid substitutions are at an amino acid residue corresponding to amino acid residues 203, 204, 206, 207, 209, 234, 235, 236, 239 and/or 277 of SEQ ID NO: 1

13. An isolated lyssavirus P-protein according to claim 12, wherein the one or more amino acid substitutions are at an amino acid residue corresponding to amino acid residues 203, 204, 206, 207, 209, 234, 235, 236, 239 and/or 277 of SEQ ID NO: 1, wherein the substitutions are 203A, 206G, 207A, 209A, 234A, 235A, 235K, 236A, 239A, and/or 277A.

14. An isolated lyssavirus P-protein according to any one of claims 1 to 13, wherein the P protein comprises at least two amino acid substitutions.

15. An isolated lyssavirus P-protein according to claim 14, wherein the at least two amino acid substitutions are at amino acid residues selected from the group consisting of amino acid residues corresponding to amino acid residues 206, 209 and 235 of SEQ ID NO: 1.

16. An isolated lyssavirus P-protein according to claim 15, wherein the two amino acid substitutions are selected from the group consisting of F209A, D235A, A206E, D235K and D236A, or an equivalent conserved position.

17. An isolated lyssavirus P-protein according to any one of claims 1 to 6 or 8 to 16, further comprising one or more amino acid substitutions in the W-hole of the P-protein.

18. An isolated lyssavirus P-protein according to claim 17, wherein one or more amino acid substitutions are at an amino acid residue corresponding to amino acid residue 265 and/or or 287 of SEQ ID NO: 1.

19. An isolated lyssavirus P-protein according to claim 18, wherein the amino acid substitutions are 265G or 287V, or an equivalent conserved position.

20. An isolated lyssavirus P-protein according to any one of claims 1 to 19, wherein the lyssavirus is selected from the group consisting of rabies virus, Lagos bat virus, Mokola virus, Duvenhage virus, European Bat lyssaviruses 1 and 2, Irkut virus, West Caucasian bat virus and Australian bat lyssavirus.

21. An isolated nucleic acid encoding a P-protein according to any one of claims 1 to 20, or a complement thereof.

22. A cell or vector comprising a nucleic acid according to claim 21.

23. A lyssavirus genome, wherein the complement of the lyssavirus genome encodes a P-protein according to any one of claims 1 to 21.

24. A lyssavirus virion comprising a lyssavirus genome according to claim 23.

25. A lyssavirus virion comprising a lyssavirus genome according to claim 24, wherein the lyssavirus is attenuated.

26. A lyssavirus virion comprising a lyssavirus genome according to claim 23, wherein the lyssavirus is able to replicate.

27. A pharmaceutical composition comprising a lyssavirus virion according to any one of claims 24 to 26 and a pharmaceutically acceptable carrier.

28. A use of a lyssavirus virion according to any one of claims 24 to 26 in the manufacture of a medicament for treating and/or preventing lyssavirus infection in a subject.

29. A method of treating and/or preventing lyssavirus infection in a subject, said method comprising administering to the subject a therapeutically effective amount of a virion according to any one of claims 24 to 26 or a pharmaceutical composition according to claim 27.


SEQUENCE LISTING (11 sequences)

SEQ ID NO: 1 - protein, 297 aa, Lyssavirus Rabies lyssavirus (strain Nishigahara RCEH)
MSKIFVNPSAIRAGLADLEMAEETVDLINRNIEDNQAHLQGEPIEVDSLPEDMSRLHLDDGKLPDLGRMSKAGEGRHQEDFQMDEGEDPSLLFQSYLDNVGVQIVRQMRSGERFLKIWSQTVEEIISYVTVNFPNPSGRSSEDKSTQTTSQEPKKETTSTPSQRKSQSLKSRTMAQTASGPPSLEWSATNEEDDLSVEAEIAHQIAESFSKKYKFPSRSSGIFLYNFEQLKMNLDDIVKEAKNVPGVTRLAHDGSKLPLRCVLGWVALANSKKFQLLVEANKLNKIMQDDLNRYASC

SEQ ID NO: 2 - protein, 297 aa, Lyssavirus Australian bat lyssavirus (isolate Human/AUS/1998)
MSKIFVNPSAIRAGMADLEMAEETVDLINRNIEDNQAHLQGEPIEVDSLPEDIKKLDISEGRSKSLVDNPQDVECRMSEDFQMDEVEDPNIQFQSYLDNIGIQIVRKMRTGERFFKIWSQTVEEIISYVGVNFPSQSGKTTENKSTQTTPKKVKTEPSSTPAKRSDQLSKTEMAAKTASGPPALEWSTTNDEDDVSVEAEIAHQIAESFSKKYKFPSRSSGIFLYNFEQLKMNLDDIVKEAKSVPGVTSLARDGLRLPLRCILGWVGSSHSKKFQLLVGSEKLNKIMQDDLNRYMSC

SEQ ID NO: 3 - protein, 298 aa, Lyssavirus European bat lyssavirus 1 (strain Bat/Germany/RV9/1968)
MSKIFVNPSALRSGLADLEMAEETVDLVNKNMEDSQAHLQGIPIDVETLPEDIKRLRIADYKQGQQEEDASRQEEGEDEDFYMTESENSYVPLQSYLDAVGMQIVRKMKTGDGFFKIWAQAVEDIVSYVATNFPAPVNKLQADKSTRTTLEKVKQAASSSAPSKREGPSSNMNLDSQESSGPPGLDWAASNDEDDGSIEAEIAHQIAESFSKKYKFPSRSSGIFLWNFEQLKMNLDDIVREVKGIPGVTRMARDGMKLPLRCMLGSVASNHSKRFQILVNSAKLGKLMQDDLNRYLAY

SEQ ID NO: 4 - protein, 297 aa, Lyssavirus European bat lyssavirus 2 (strain Human/Scotland/RV1333/2002)
MSKIFVNPSAIRAGLADLEMAEETVDLVNKNIEDNQAHLQGEPIEVDALPEDMSKLQISERRPAQFTDNTGGKEEGSDEDFYMAESEDPYIPLQSYLEGVGIQLVRQMKTGERFFKIWSQAVEEIISYVTVHFPMPLGKSTEDKSTQTPEEKFKPSPQQAVTKKESQSSKIKTISQESSGPPALEWSTTNDEENASVEAEIAHQIAESFSKKYKFPSRSSGIFLFNFEQLKMNLDDIVKEAKKIPGVVRLAQDGFRLPLRCILGGVGSVNSKKFQLLVNSDKLGKIMQDDLNRYLAY

SEQ ID NO: 5 - protein, 298 aa, Lyssavirus Duvenhage virus
MSKIFINPSDIRSGLADLEMAEETVELVNRNMEDSQAHLQGVPIDVETLPEDIQRLHITDPQASLRQDMVDEQKHQEDEDFYLTGRENPLSPFQTHLDAIGLRIVRKMKTGEGFFKIWSQAVEDIVSYVALNFSIPVNKLFEDKSTQTVTEKSQQASASSAPNRHEKSSQNARVNSKDASGPAALDWTASNEADDESVEAEIAHQIAESFSKKYKFPSRSSGIFLWNFEQLKMNLDEIVREVKEIPGVIKMAKDGMKLPLRCMLGGVASTHSRRFQILVNPEKLGKVMQEDLDKYLTY

SEQ ID NO: 6 - protein, 298 aa, Lyssavirus Irkut virus
MSKIFVNPSAIRAGLADLEMAEETIDLINRTIEDNQAHLQGVPIEVEALPEDMKKLQISDHQQGQPSGGATGQDGSEEEDFYMTESENPYIPFQSYLDAVGIQLVRKMKTGEGFLKIWSQAAEEIVSYVAINFPLPADKESAEKSTQTVGEPLKSNSASNTPNKRSKPSTSTDLKAQEASGPHGIDWAASNDEDDASVEAEIAHQIAESFSKKYKFPSRSSGIFLWNFEQLKMNLDDIVGGAKEIPGVIRMAKEGNKLPLRCILGGVALTHSKRFQVLVNSEKLGRIMQEDLNKYLAN

SEQ ID NO: 7 - protein, 297 aa, Lyssavirus Aravan virus
MSKIFVNPSAIRAGLADLEMAEETVDLVNKNVEESQAHLQAEPIEVDALPEDMKRLQISEPKPCQLPDGTCMKEEGGDEDFYMAESGDPYIPLQSYLDTMGIQIVRKMKTGERFFKIWSQSVEEIISYVAVNFPVPPGKSLADKSTQTSVEKSKPASQPTQPKKEDQLSKVNIDSQESSGPPALDWAATNDDDDASVEAEIAHQIAESFSKKYKFPSRSSGIFLYNFEQLKMNLDDIVREAKGIPGVTRRAGDGVRLPLRCILGWVASTHSRRFQLLVNSDKLNKVMQDDINRYLAY

SEQ ID NO: 8 - protein, 297 aa, Lyssavirus Khujand virus
MSKIFVNPSAIRAGLADLEMAEETVDLINRNVEDNQAHLQGEPIEVEALPEDMRRLHISEQKHSQLSDSACGKEEGSDDDFYMADSEDPYVPMQSYLDNVGIQIVKKMKTGERFFKIWSQAVEEIISYVTVNFPLPSGKSTDDKSTQTVSERSRQNPQPSSVKKEDQLSKTKVVSQEASGPPALEWSATNDEDDASVEAEIAHQIAESFSKKYKFPSRSSGIFLYNFEQLKTNLDDIVREAKRIPGVMRLAQDGLRLPLRCILGWVASTHSKRFQILVDSDKLSKIMQDDINRYLAY

SEQ ID NO: 9 - protein, 303 aa, Lyssavirus Mokola virus
MSKDLVHPSLIRAGIVELEMAEETTDLINRTIESNQAHLQGEPLYVDSLPEDMSRLRIEDKSRRTKTEEEERDEGSSEEDNYLSEGQDPLIPFQNFLDEIGARAVKRLKTGEGFFRVWSALSDDIKGYVSTNIMTSGERDTKSIQIQTEPTASVSSGNESRHDSESMHDPNDKKDHTPDHDVVPDIESSTDKGEIRDIEGEVAHQVAESFSKKYKFPSRSSGIFLWNFEQLKMNLDDIVKAAMNVPGVERIAEKGGKLPLRCILGFVALDSSKRFRLLADNDKVARLIQEDINSYMARLEEAE

SEQ ID NO: 10 - protein, 305 aa, Lyssavirus Lagos bat virus
MSKGLIHPSAIRSGLVDLEMAEETVDLVHKNLADSQAHLQGEPLNVDSLPEDMRKMRLTNAPSEREIIEEDEEEYSSEDEYYLSQGQDPMVPFQNFLDELGTQIVRRMKSGDGFFKIWSAASEDIKGYVLSTFMKPETQATVSKPTQTDSLSVPRPSQGYTSVPRDKPSNSESQGGGVKPKKVQKSEWTRDTDEISDIEGEVAHQVAESFSKKYKFPSRSSGIFLWNFEQLKMNLDDIVKTSMNVPGVDKIAEKGGKLPLRCILGFVSLDSSKRFRLLADTDKVARLMQDDIHNYMTRIEEIDHN

SEQ ID NO: 11 - protein, 297 aa, Lyssavirus Caucasian bat virus
MSKSLIHPSDLRAGLADIEMADETVDLVYKNLSEGQAHLQGEPFDIKDLPEGVSKLQISDNVRSDTSPNEYSDEDDEEGEDEYEEVYDPVSAFQDFLDETGSYLISKLKKGEKIKKTWSEVSRVIYSYVMSNFPPRPPKPTTKDIAVQADLKKPNEIQKISEHKSKSEPSPREPVVEMHKHATLENPEDDEGALESEIAHQVAESYSKKYKFPSKSSGIFLWNFEQLKMNLDDIVQVARGVPGISQIVERGGKLPLRCMLGYVGLETSKRFRSLVNQDKLCKLMQEDLNAYSVSSNN